(19) United States
(12) Patent Application Publication
(10) Pub. No.: US 2007/0196364 A1
(43) Pub. Date: Aug. 23, 2007

(54) Pharmaceutical Formulation and Process

(75) Inventors: Rajesh Krishnamurthy, Germantown, MD (US); Raj Suryanarayanan, Roseville, MN (US); Xiangmin Liao, Commack, NY (US)

Correspondence Address: HUMAN GENOME SCIENCES INC.;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 11/626,264

(22) Filed: Jan. 23, 2007

Related U.S. Application Data
(63) Continuation-in-part of application No. PCT/US05/26506, filed on Jul. 27, 2005.
(60) Provisional application No. 60/746,585, filed on May 5, 2006.
(60) Provisional application No. 60/591,102, filed on Jul. 27, 2004.
(60) Provisional application No. 60/677,838, filed on May 5, 2005.

(51) Int. Cl. A61K 39/395; A61K 38/17; A61K 31/7012

(52) U.S. Cl. 424/133.1; 514/12; 514/53

(57) ABSTRACT

A process for lyophilization or freeze-drying of a pharmaceutical product is provided and a liquid formulation suitable for lyophilization. In particular, a process for lyophilization or freeze-drying a liquid formulation that includes a protein active agent, a bulking agent and a saccharide stabilizing agent is provided. The saccharide to bulking agent ratio and the protein concentration of the formulation are important factors that affect crystallization of the bulking agent during lyophilization and storage as are some processing conditions. In one embodiment, the saccharide is a disaccharide, such as sucrose and the crystalline bulking agent is mannitol. The protein can be an antibody or a non-antibody protein.

### RELATED APPLICATIONS

This application claims benefit under 35 U.S.C. §119(e) of U.S. Provisional Application No. 60/746,585, filed May 5, 2006; this application also is a continuation-in-part of International Application No. PCT/US2005/026506, filed Jul. 27, 2005, which claims benefit under 35 U.S.C. §119(e) of U.S. Provisional Application No. 60/591,102, filed Jul. 27, 2004 and U.S. Provisional Application No. 60/677,838, filed May 5, 2005, each of which is hereby incorporated by reference in its entirety.

### FIELD OF THE INVENTION

The invention relates to the lyophilization or freeze-drying of a liquid formulation. More particularly, the invention provides an improved process for lyophilization or freeze-drying a liquid pharmaceutical formulation that includes a protein active agent.

### BACKGROUND OF THE INVENTION

The stability and/or potency of many pharmaceutical and food products can be adversely affected during long-term storage. Loss of potency may be attributable to direct chemical degradation or structural alteration. Examples of degradative chemical reactions include, but are not limited to, hydrolysis, oxidation, isomerization, deamidation, disulfide scrambling, and racemization. Examples of structural alterations include, but are not limited to, denaturation, aggregation, precipitation and polymerization.

Lyophilization (also called freeze-drying) refers to a process that uses low temperature and pressure to remove a solvent, typically water, from a liquid formulation by the process of sublimation (i.e., a change in phase from solid to vapor without passing through a liquid phase). Lyophilization helps stabilize pharmaceutical formulations by reducing the solvent component or components to levels that no longer support chemical reactions or biological growth. Since drying during lyophilization takes place at a low temperature, chemical decomposition is also reduced. Additionally, freeze dried products have a high specific surface area, which may enhance product dissolution during reconstitution.

Conventionally, crystalline bulking agents such as mannitol or glycine have been included in freeze-dried formulations. Mannitol is commonly used as a bulking agent. It readily forms a crystalline cake and has a high eutectic melting temperature in the presence of ice which facilitates primary drying at a relatively high temperature. Crystalline mannitol also provides a robust matrix during freeze-drying, which reduces the likelihood of “micro-collapse” of the amorphous content (i.e., protein) of the solution.

Mannitol can exist in numerous physical forms including, but not limited to, three anhydrous polymorphs (α-, β-, δ-), mannitol hemihydrate and amorphous mannitol. The physical form of mannitol in the final lyophile may affect stability of the active agent. Among the anhydrous polymorphs, β-mannitol is stable under ambient conditions. The hemihydrate form of mannitol is generally unstable in a freeze-dried formulation and thus its presence may affect product stability. For example, crystallized mannitol hemihydrate present in the final lyophile may undergo dehydration during storage in which sorbed water is released and becomes available to interact with the active agent which may cause decreased stability.

Studies have shown that low mannitol concentration (1.5% and 3%) in the prelyophilized solution tend to favor the β-mannitol form, while higher concentration tend to favor α-mannitol forms. Furthermore, it was shown that a slow cooling rate resulted in a mixture of δ-(majority) and α-mannitol (minority) forms while rapid cooling rates resulted in a mixture of α-(majority) and δ-mannitol (minority) forms. See, Cannon et al., PDA J. Pharm. Sci. Tech. 54:13-22 (2000). However, an extra annealing step (−20° C.) was able to substantially reduce both α- and δ-mannitol forms and resulted in an increased amount of the more stable β-form.

Crystallization of bulking agents during lyophilization or storage can result in reduced product stability. Nonetheless, mannitol can be retained in the amorphous state if other components of the formulation inhibit mannitol crystallization. Publications have shown that lyoprotectants such as sucrose and trehalose can inhibit mannitol crystallization depending on their concentrations. See, Kim et al., J. Pharm. Sci. 87:931-935 (1998). Additional publications have investigated the effect of lyoprotectants (such as sucrose or trehalose), alone or in combination with bulking agents such as mannitol, on the storage stability of protein formulations. See, Cleland et al., J. Pharm. Sci. 90(3):310-321 (2001). Izutzu et al., Chem. Pharm. Bull. 42(1):5-8 (1994) investigate the effect of mannitol crystallization on protein activity.

However, an evaluation of multiple formulation components and/or processing conditions in the presence of the active agent resulting in the crystallization of bulking agents would be beneficial if performed. Therefore, there remains a need to systematically determine the effect of multiple formulation components and/or processing conditions on the stability of a protein formulation.

### SUMMARY OF THE INVENTION

The invention provides a liquid formulation suitable for freeze-drying to form a freeze-dried formulation. In one embodiment, the liquid formulation includes at least a protein active agent, a saccharide stabilizing agent, and a bulking agent. According to the invention, the ratio of bullring agent to saccharide stabilizing agent and protein active agent in the liquid formulation is sufficient to maintain the bulking agent in a substantially amorphous state, i.e., in which less than about 49 wt. % of the bulking agent present in the formulation is in a crystalline state. In one embodiment, the bullring agent is less than 25 wt %, 15 wt %, 10 wt %, 7 wt %, or 5 wt % crystalline. In one embodiment, the saccharide stabilizing agent is a disaccharide, such as sucrose and the bulking agent is mannitol. The protein active agent can be an antibody or a non-antibody protein.

In another embodiment, the liquid formulation includes at least a protein active agent, a saccharide stabilizing agent, a bullring agent, and a nonionic surfactant. In one embodiment, the nonionic surfactant is included in the liquid formulation at or above the CMC for the surfactant. In another embodiment, the nonionic surfactant is included in the liquid formulation below the CMC for the surfactant, for example, at less than 10%, 20%, 30%, 40%, 50%, 60%, 70%, 80%, or 90% of the CMC of the surfactant. In another embodiment, the nonionic surfactant is added in an amount between 30% and 70%, 40% and 60%, or 45% and 55% of the CMC of the surfactant.

The invention also provides a process for freeze-drying a liquid formulation that includes a protein active agent, a saccharide stabilizing agent and a bulking agent. The freeze-drying process comprises steps of freezing, annealing, primary drying and secondary drying under various conditions such that the bulking agent is maintained in a substantially amorphous state or is maintained in a substantially crystalline state. In one embodiment, the freeze-drying process conditions are modified to reduce the formation of mannitol hemihydrate in the final lyophile. The freeze-drying process conditions can be determined based on the concentration of the active agent present in the liquid formulation and the effect of the active agent on the formation of mannitol hemihydrate.

### BRIEF DESCRIPTION OF THE FIGURES

FIG. 1 shows a typical phase diagram of an element or a simple compound. The stability of solid, liquid and gas phases depends on the temperature and the pressure. The three phases are in equilibrium at the triple point. The gas and liquid phases are separated by a phase transition only below the temperature of the critical point. It is possible to change continuously between the two phases at higher temperatures. Only the solid phase exists at the absolute zero of temperature (0 K). There are generally several phases within the solid phase corresponding to different crystal symmetries. For mixtures of two or more elements the phase diagrams also depend on the concentrations of the elements.

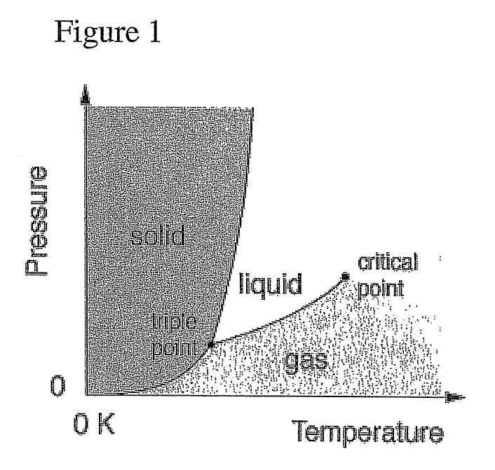

FIG. 2 shows the effect of mannitol to sucrose ratio on Tg′ (no protein).

FIG. 3 shows the effect of mannitol to sucrose ratio on Tg′ in the presence of 20 mg/ml protein.

FIG. 4 shows the effect of protein concentration on Tg′ (mannitol to sucrose ratio of 1.1).

FIG. 5 shows the effect of the protein concentration on Tg′ (mannitol to sucrose ratio 1.1).

FIG. 6 shows the effect of the annealing temperature on mannitol crystallization (mannitol to sucrose ratio of 3.0; no protein).

FIG. 7 shows the effect of the annealing time on mannitol crystallization (mannitol to sucrose ratio of 3.0; no protein).

FIG. 8 shows the effect of the annealing temperature on mannitol crystallization (mannitol to sucrose ratio 1.95, no protein).

FIG. 9 shows the effect of the annealing time on mannitol crystallization (mannitol to sucrose ratio 1.95, no protein).

FIG. 10 shows the real time monitoring of mannitol crystallization during freeze-drying by in situ XRD (mannitol to sucrose ratio of 3.0; no protein).

FIG. 11 shows the real-time monitoring of mannitol crystallization during freeze-drying by in situ XRD (mannitol to sucrose ratio of 3.0; 11 mg/ml protein).

FIG. 12 shows the effect of changing protein concentration on mannitol crystallization in a 3:1 mannitol to sucrose solution. The arrow indicates a protein concentration in which the mannitol is substantially amorphous.

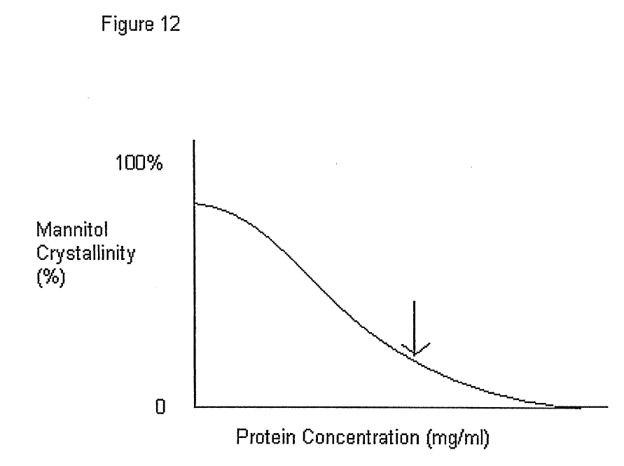

FIG. 13 shows DSC heating profiles of frozen aqueous mannitol-sucrose solutions. The solutions were initially cooled from room temperature to −70° C. at 20° C./min, held at −70° C. for 30 min and heated to room temperature at 5° C./min. The glass transition regions are expanded in the inset.

FIG. 14 shows the effect of mannitol to sucrose ratios on the Tg′. The solutions were cooled from room temperature to −70° C. at 20° C./min, held at −70° C. for 30 min and heated to room temperature at 5° C./min. The protein concentration was 20 mg/ml. Each point is the mean of three determinations. Error bars represent standard deviations (n=3).

FIG. 15 shows DSC heating profiles of frozen aqueous mannitol-sucrose and 5% mannitol-only solutions. The solutions were initially cooled from room temperature to −70° C. at 20° C./min, held for 30 min and heated to room temperature at 5° C./min.

FIG. 16 shows the effect of protein concentrations on the Tg′. The solutions were cooled from room temperature to −70° C. at 20° C./min. The solutions were held at −70° C. for 30 min and heated to room temperature at 5° C./min. The mannitol to sucrose weight ratios (R) were 0.45, 1.5 and 3.0, respectively. Each point is the mean of three determinations. Error bars represent standard deviations (n=3).

FIG. 17 shows the inhibitory effect of protein concentrations on mannitol crystallization. The solutions were cooled from room temperature to −70 ° C. at 20° C./min, held held at −70° C. for 30 min and heated to room temperature at 5° C./min. The mannitol to sucrose weight ratio (R) was 3.0. The arrows show the trend in the crystallization onset temperature as a function of the protein concentration.

FIG. 18 shows the effect of protein concentration on the enthalpy of crystallization as a function of annealing time. The solutions were cooled from room temperature to −70° C. at 20° C./min., annealed at −45° C. and then heated to room temperature at 5° C./min. The mannitol to sucrose weight ratio was 3.0. Each point is the mean of three determinations. Error bars represent standard deviations (n=3).

FIG. 19 shows the effect of annealing temperature on the crystallization behavior of mannitol in frozen aqueous (A) mannitol-sucrose and (B) mannitol-sucrose-protein solutions. The solutions were cooled from room temperature to −70° C. at 20° C./min. The solutions were held at −70° C. for 30 min and heated to the annealing temperature at 5° C./min, annealed for 60 minutes and cooled back to −70° C. The solutions were reheated to room temperature at 5° C./min. The second heating scans are shown here. The mannitol to sucrose weight ratio was 3.0 and the protein concentration was 20 mg/ml.

FIG. 20 shows DSC heating profiles of frozen aqueous mannitol-sucrose solutions in the absence and the presence of the protein. The solutions were cooled from room temperature to −70° C. at 20° C./min. The solutions were annealed at −35° C. and then heated to room temperature at 5° C./min. The mannitol to sucrose ratio was fixed at 3.0. The protein concentration was 20 mg/ml.

FIG. 21 shows XRD patterns of frozen aqueous mannitol-sucrose solutions (A) in the absence and (B) the presence of the protein. The protein concentration was 20 mg/ml. (I) The solutions were cooled from room temperature to −70° and XRD pattern was obtained. (II) The temperature was raised to −45°, annealed for 1 hour. (III) In order to remove the thermal history, the sample was heated to room temperature, cooled back to −70°, temperature was then raised to −35° and annealed for 1 hour. (IV) After again heating to room temperature and cooling back to −70°, the temperature was raised to −25° and annealed for 15 minutes (in the absence of protein) and 1 hour (in the presence of protein). All heating and cooling rates were 5 and 10° C./min, respectively.

FIG. 22 shows XRD patterns of lyophiles obtained from a prelyo solution containing 2.5 mg/mL protein, 200 mM mannitol and 60 mM trehalose in 10 mM phosphate buffer (pH=7.2). The prelyo solutions were processed under different conditions. (A) Annealed at −18° C. for 1 hour, primary dried at −5° C. for 14 hours and secondary dried at 35° C. for 10 hours. (B) Annealed at −18° C. for 5 hours, primary dried at 5° C. for 12 hours and secondary dried at 45° C. for 10 hours. A characteristic peak of mannitol hemihydrate (★) and δ-mannitol (▴) are pointed out.

FIG. 23 shows DSC heating curves of lyophiles A and B. (Bottom) TGA and DTGA curves of lyophile A. For the DSC analyses, the samples were cooled from room temperature to −40° C., and then heated to 200° C., both at 10° C./min. For the TGA analysis, the sample was heated from 35 (° C.) to 200° C. at 10° C./min.

FIG. 24 shows XRD patterns of frozen solution cooled at (a) 10° C./min and (b) 1° C./min from room temperature to −70° C. The solutions contained 1.7 mg/mL protein, 200 mM mannitol and 60 mM trehalose in 10 mM phosphate buffer (pH=7.2). The peaks at 22.5, 24.2 and 25.6° 2θ can be attributed to hexagonal ice.

FIG. 25 shows DSC heating curves of a frozen prelyo solution (a) containing 1.7 mg/ml protein and (b) placebo. The solutions were initially cooled from room temperature to −70° C. at 20° C./min, held for 30 minutes, and heated to room temperature at 5° C./min. The mannitol, trehalose and phosphate buffer concentrations were 200, 60 and 10 mM, respectively. The crystallization onset temperature of mannitol is pointed out.

FIG. 26 shows the effect of annealing temperature on mannitol crystallization. DSC heating curves of a frozen prelyo solution containing 1.7 mg/ml protein. The solutions were initially cooled from room temperature to −70° C. at 20° C./min, held for 30 minutes, heated to −28° C. at 5° C./min and annealed for (a) 0, (b) 15, (c) 30 and (d) 60 minutes. The solutions were then cooled to −65° C. at 20° C./min, held for 15 minutes, heated to room temperature at 5° C./min. The second heating scans are shown. The mannitol, trehalose and phosphate buffer concentrations were 200, 60 and 10 mM, respectively. The line shows the trend in the crystallization exotherm of mannitol as a function of annealing time. The Tg′ is also pointed out.

FIG. 27 shows the effect of annealing temperature on mannitol crystallization. DSC heating curves of frozen prelyo solution containing 1.7 mg/ml protein. The solutions were initially cooled from room temperature to −70° C. at 20° C./min, held for 30 minutes, heated to −18° C. at 5° C./min and annealed for (a) 0, (b) 15, (c) 30 and (d) 60 minutes. The solutions were then cooled to −65° C. at 20° C./min, held for 15 minutes, heated to room temperature at 5° C./min. The second heating scans are shown. The mannitol, trehalose and phosphate buffer concentrations were 200, 60 and 10 mM, respectively. Crystallization of mannitol (exotherm) was evident only in the unannealed system. The Tg′ is pointed out, and is observed to shift to a higher temperature as the annealing time is increased.

FIG. 28 shows XRD patterns of frozen aqueous prelyo solutions (a) containing 1.7 mg/ml protein and (b) placebo. The mannitol, trehalose and phosphate buffer concentrations were 200, 60 and 10 mM, respectively. The solutions were initially cooled from room temperature to −45° C. at 1° C./min, heated to −18° at 1° C./min, and annealed for 3 hours. A characteristic peak of mannitol hemihydrate (*) and δ-mannitol (★) are pointed out.

FIG. 29 shows the effect of the protein on mannitol phases crystallizing from solution as a function of annealing time. (1) Protein solution annealed at −8° C., (2) placebo solution annealed at −8° C., (3) protein solution annealed at −18° C., and (4) placebo solution annealed at −18° C. The prelyo solutions were initially cooled at 1° C./min from room temperature to −45° C., and then heated to selected annealing temperatures at 1° C./min. The protein, mannitol, trehalose and phosphate buffer concentrations were 1.7 mg/ml, 200, 60 and 10 mM, respectively. *(intensity of 20.4° 2θ peak of δ-mannitol/intensity of 17.9° 2θ peak of mannitol hemihydrate)

FIG. 30 shows XRD patterns of frozen aqueous prelyo solutions (a) containing 1.7 mg/ml protein and (b) placebo. The mannitol, trehalose and phosphate buffer concentrations were 200, 60 and 10 mM, respectively. The solutions were initially cooled from room temperature to −45° C. at 1° C./min. The temperature was raised to −80 at 1° C./min, annealed for 3 hours. One characteristic peak of mannitol hemihydrate (*) and δ-mannitol (★) are pointed out.

FIG. 31 shows XRD patterns of protein lyophiles primary dried for 1 hour at (a) −5° C. after annealing at −18° C., (b) −5° C. after annealing at −8° C., and (c) at −20° after annealing at −8° C. The protein, mannitol and trehalose concentrations were 1.7 mg/ml, 200 mM and 60 mM, respectively. The solutions were initially cooled from room temperature to −45° C. at 1° C./min. The temperature was raised to the desired annealing temperatures at 1° C./min, annealed for 3 hours. Primary drying was conducted at a chamber pressure ˜100 mTorr. The characteristic peaks of mannitol hemihydrate (*) and δ-mannitol (★) are marked.

FIG. 32 shows XRD patterns of protein lyophiles after secondary drying for 30 minutes at (I) 25° C., (II) 45° C., and (III) 65° C. The protein, mannitol and trehalose concentrations were 1.7 mg/mL, 200 mM and 60 mM, respectively. The solutions were initially cooled from room temperature to −45° C. at 1° C./min. The temperature was raised to −18° at 1° C./min, annealed for 3 hours. The annealed solution was heated to −5° C. and subjected to primary drying for 1 hour at a chamber pressure ˜100 mTorr. The characteristic peak,s of mannitol hemihydrate (*) and δ-mannitol (★) are marked.

### DETAILED DESCRIPTION OF THE INVENTION

To promote a better understanding of the invention, Applicants will first provide a discussion of lyophilization in general.

I. Lyophilization in General

Lyophilization (also called freeze-drying) refers to a process that uses low temperature and pressure to remove a solvent, typically water, from a liquid formulation by the process of sublimation (i.e., a change in phase from solid to vapor without passing through a liquid phase). Lyophilization helps stabilize pharmaceutical formulations by reducing the solvent component or components to levels that no longer support chemical reactions or biological growth. Since drying during lyophilization takes place at a low temperature, chemical decomposition is also reduced.

Freeze-drying processes are known. In some instances, freeze-drying is performed in a “manifold” process in which flasks, ampules or vials are individually attached to the ports of a manifold or drying chamber. In other instances, freeze-drying is performed as a “batch” process in which one or more similar sized vessels containing like products are placed together in a tray dryer. In a “bulk” process, the product is poured into a bulk pan and dried as a single unit. Product is removed from the freeze dry system prior to closure and then packaged in air tight containers. The invention described herein can be used in combination with any of these or other known methods.

Generally, lyophilization takes place in at least three stages: freezing; primary drying; and secondary drying. In some instances, it may be desirable to include an annealing step between the freezing and primary drying stages. Each of these stages will be discussed in more detail below.

Freezing

During the freezing process, a liquid solution or formulation that contains at least one solvent and at least one solute is placed in a container, which is then placed in a freeze-dryer. The primary goal of the freezing process is to solidify at least the solvent component of the formulation. In the freezing process, the liquid formulation is therefore cooled to a sufficiently low temperature to allow for solidification of at least the solvent component.

During the freezing process, the microstructure of both the solvent crystals and the solute is formed. This microstructure can affect both the quality of the final product and its processing characteristics, such as the rates of primary and secondary drying. If both the solute and the solvent crystallize during the freezing process, the temperature at which the formulation becomes solid is called the eutectic temperature (Te). A formulation in which both the solute and solvent crystallize during the freezing process is referred to herein as “a crystalline system.” If some or all of the solute remains substantially amorphous during the freezing process, the temperature at which the solute becomes a glassy or amorphous solid is called the glass transition temperature (Tg′). A formulation in which at least some of the solute remains in an amorphous state is referred to herein as “an amorphous system.” One example of an amorphous system is a liquid formulation that contains protein as an active agent. As the temperature of an “amorphous system” is reduced below Tg′, the solvent component forms crystals (referred to as “the crystalline component”). The crystalline component may also contain crystalline excipients, for example bulking agents such as mannitol or glycine. The concentration of the solute that remains amorphous (herein referred to as the “amorphous component”) increases as the temperature of the formulation is decreased and the solvent crystallizes out of solution. Typically, the amorphous component includes the amorphous active agent, for example, a protein, and any amorphous excipients, for example, saccharide stabilizing agents. It is worthwhile to note that an element of a formulation, such as a bulking agent, can exist in a crystalline or an amorphous state depending upon the formulation and the processing parameters. The freezing process separates the amorphous component from the crystalline component. Consequently, as used herein, a “frozen” amorphous system contains a crystalline component, which can include the crystalline solvent and crystalline excipients; and an amorphous component located in the interstitial regions of the crystalline component. The amorphous component can include the amorphous active agent; one or more amorphous excipients; and any remaining unfrozen solvent.

During the freezing process solvent molecules may spontaneously aggregate to form a template to which other solvent molecules can attach and ultimately form a crystal. This process is referred to as “nucleation.” The temperature at which nucleation occurs (“the nucleation temperature”) can affect primary drying rate and morphology. As the temperature decreases, the probability of nucleation temporarily increases. However, as the temperature is decreased further, nucleation tends to decrease due to the increased viscosity of the system. The nucleation observed in pharmaceutical solutions is largely a heterogeneous nucleation. The nucleation temperature can be affected by environmental particulates, freezing method and the presence or absence of nucleating agents. As used herein, the term “heterogeneous” nucleation refers to nucleation that was initiated by foreign particles (also called nucleation sites) in the solution or on the surface of the container in which the solution was placed. The term “homogenous nucleation” refers to nucleation that occurs in the absence of a nucleation site in the solution. Generally, homogeneous nucleation is caused by the aggregation of slow moving molecules.

The rate and method of cooling can influence the structure and appearance of the matrix and final product, including whether an excipient, such as a bulking agent, is substantially crystalline or substantially amorphous. For example, if the solution is frozen quickly, the crystals will tend to be small. This may result in a fine pore structure in the product and a corresponding higher resistance to flow of water vapor during primary drying and hence, a longer primary drying time. However, small crystal structures may be desirable, for example, to preserve structures for microscopic examination. If the solution is frozen more slowly, the crystals will tend to grow from the cooling surface and may be larger. As a result, the resulting product may have a coarse pore structure, resulting in less restrictive channels in the matrix and perhaps resulting in a shorter primary drying time.

Supercooling is another physical event that is observed during the freezing process (in addition to solvent crystallization; concentration of solutes; and in some cases, crystallization of the solute). The term “supercooling” refers to the reduction of the temperature of a liquid beyond its freezing point. For example, supercooled water is water that remains in a liquid state when it is at a temperature that is well below freezing. As a result of supercooling, the product temperature may have to be decreased significantly below the actual freezing point of the solution before freezing occurs.

Although the temperature at which the material is frozen depends on many factors, including the formulation, the freezing process is typically performed at a temperature between below at least about 0° C., more typically below at least about −10° C., below at least about −25° C., below at least about −40° C. or below at least about −50° C. Typically the freezing process is performed at atmospheric pressure.

Annealing

Because the nucleation temperature may affect the primary drying rate and resulting cake morphology, it may be desirable to control the nucleation temperature. One method by which the nucleation temperature dependence can be circumvented is by introducing a post-freezing annealing step in the lyophilization process. An annealing process results in the removal of solvent crystals smaller than a critical size and generation of larger solvent crystals. The increased crystal size results in an increased primary drying rate because the pores in the material left by the large crystals provide less resistance to primary drying than smaller pores left by smaller crystals. Annealing also reduces freezing-induced drying rate heterogeneity—i.e., product differences between vials in the same batch. Annealing is particularly beneficial for crystallizing excipients and bulking agents present in the formulation. In particular, annealing promotes crystallization of bulking agents such as mannitol. Annealing can increase the primary drying rate of a frozen formulation between about 1 and 5 fold.

In general, an annealing process involves maintaining a sample below its freezing point or Tg′ for a predetermined period of time. In some instances, it may be desirable to perform the annealing process at a temperature that is above the Tg′ of the material, for example, to increase the rate of the annealing process. Because the solvent is already substantially frozen prior to the annealing process, and because, unlike in primary drying, the solvent is not being removed, the annealing process can be performed at a relatively high temperature without melting the cake. Although the processing parameters of the annealing step depend on the formulation, the formulation is generally held at a temperature between about −50° C. and about 0° C. for a time ranging between a few minutes to a few days, typically between about 15 minutes and 24 hours, or between about 15 minutes and 10 hours. It is believed that the annealing process increases crystal size of the bulking agent and simplifies amorphous structure, resulting in larger and more numerous pores in the annealed samples.

Primary Drying

During the primary drying process, the solvent is removed from the liquid formulation by a process of sublimation. As used herein, the term “sublimation” refers to the transition of a solid to a gas, without passing through a liquid stage.

One goal of the primary drying process is to remove all of the “mobile” solvent from the formulation. Although a majority (i.e., at least about 50 wt %) of the solvent is removed from the formulation during primary drying, the formulation that remains at the end of the primary drying process includes an amorphous component within a glassy matrix that contains between about 5 wt % and about 49 wt %, more typically between about 10 wt % and about 40 wt %, or between about 10 wt % and about 20 wt % solvent. Although the composition of the liquid formulation can affect the cycle time, as well as the desired amount of solvent in the final product, primary drying typically takes between about 10 hours and about 10 days, more typically between about 1 day and about 4 days.

In general, the primary drying process is performed at a reduced pressure (i.e., vacuum) and at a temperature higher than the temperature at which the system was frozen. The increase in temperature provides energy for sublimation. However, it is important to control the drying rate and the heating rate during primary drying. If the drying proceeds too rapidly, the dried product can be carried out of the container by escaping solvent vapor. If the amorphous system is heated too rapidly, it may melt or collapse, causing degradation of the final product and/or changing the physical characteristics of the dried material, for example, making it visually unappealing and harder to reconstitute. Increased time for reconstitution at the user stage may result in partial loss of potency if the drug is not completely dissolved, since it is common to use in-line filters during administration to the patient. If the cake collapses, solvent can be trapped in the cake, which may result in product instability. However, primary drying at lower temperatures tends to increase cycle time, which usually results in a more expensive process. Additionally, process parameters used during primary drying can affect the state of formulation components. For example, for a particular formulation, a bulking agent may exist in a crystalline or an amorphous state depending upon the processing parameters.

Temperature

The term “temperature” can refer to the “shelf temperature” or the “product temperature.” As used herein, the term “shelf temperature” refers to the temperature of the lyophilization equipment. The “product temperature” refers to the actual temperature of the formulation. Although the “shelf temperature” affects the product temperature (and thus the cycle time of the lyophilization process), the shelf temperature and the product temperature can differ for a variety of reasons. For example, sublimation is an endothermic process. Therefore, as the solvent sublimes, the remaining amorphous component of the formulation tends to cool. Therefore, throughout primary drying, the amorphous component of the formulation tends to remain colder than the shelf temperature. At the end of primary drying, when the mobile solvent has been removed by sublimation, and the heat of sublimation is no longer needed, the temperature of the amorphous component of the formulation tends to increase sharply toward the shelf temperature. Consequently, the asymptotic rise in temperature may be used to detect the endpoint of primary drying. Other temperature-related properties of a formulation include the “eutectic temperature” (Te) and the “glass transition temperature” (Tg′).

Eutectic Temperature (Te)

The eutectic temperature refers to the temperature at which the solute and solvent present in a crystalline system become frozen or crystalline. As used herein, the term “crystalline” or “crystal” refers to a solid in which the constituent atoms, molecules or ions are packed in a regularly ordered, repeating pattern extending in all three spatial dimensions. Under some conditions, the solid may include a single crystal, where all of the atoms in the solid fit into the same lattice or crystal structure. However, it is more typical that many crystals form simultaneously during solidification, leading to a polycrystalline solid. As used herein, the term “crystalline system” refers to a mixture in which all components, i.e., both the solvent and solute, form crystals. Conventionally, the Te has been used as a guide for determining the maximum product temperature for a crystalline system during primary drying. It is generally thought that the desirable properties of a freeze-dried product may be lost if the product temperature exceeds the eutectic temperature while the frozen solvent is still present because drying will take place from the liquid state instead of the solid state.

Glass Transition Temperature (Tg′)

For solutions in which the solute does not readily crystallize during freezing (i.e., “amorphous systems”), the temperature at which the viscosity of the system changes from a viscous liquid to a glass is called the “glass transition temperature” (Tg′). More specifically, as the temperature of an amorphous system is decreased, a critical concentration is achieved at which point the unfrozen fraction exhibits a reduced molecular mobility, and its physical state changes from an elastic liquid to a brittle but amorphous solid glass. The unfrozen fraction is referred to herein as “the amorphous component.” The amorphous component is a solid, but unlike a crystalline solid, there is no long-range order of the positions of the atoms, molecules or ions. In protein-based pharmaceutical formulations, the amorphous component contains at least the protein active agent. The amorphous component may also contain one or more excipients. The glass transition temperature can be defined empirically as the temperature at which the viscosity of the liquid exceeds a certain value, for example, 10¹³ Pascal-seconds. The glass transition temperature for pure water is about −134° C. In general, the glass transition temperature of a solute is higher, and the glass transition temperature for the solution falls somewhere in between. The glass transition temperature for an amorphous system is not a single temperature point, but rather a range of temperatures, usually within a range of 1-2° C. In many instances, the viscosity of the amorphous component changes by three or four orders of magnitude over a temperature range of a few degrees at temperatures around the glass transition temperature. Additionally, below the glass transition temperature, the mobility of the amorphous component is greatly restricted and many degradative reactions are greatly slowed.

An example of an amorphous system is a protein-based formulation. As with crystalline systems, conventionally it is thought that the glass transition temperature represents the maximum allowable temperature during the primary drying of the amorphous system.

Collapse Temperature (Tc)

Another concept that is closely related to the glass transition temperature is the collapse temperature of an amorphous system. The collapse temperature refers to the temperature at which the mobility of the amorphous component in the interstitial regions of the crystalline component increases or becomes significant. Within a given system or formulation, as solvent is reduced via sublimation, the collapse temperature tends to increase.

In most amorphous systems, the onset temperature for the mobility of the amorphous phase in the interstitial region is not sharp or well defined and may occur over a range of temperatures. The collapse temperature may also be affected by the measurement method and the residual unfrozen solvent contained in the amorphous component. The collapse temperature is a function of all constituents present including the amorphous component and can therefore vary depending on the formulation.

In general, the collapse temperature may refer to the loss or disappearance of crystal structure within the crystalline component or the generation of new crystal patterns. Collapse may also refer to both the viscous flow of the amorphous component in combination with the resultant loss of the microstructure that was established by freezing. Typically, collapse is associated with a decreased surface area of the freeze-dried formulation, reduction in cake volume, and/or loss of pharmaceutical elegance. Collapse may also be associated with a glossy or glassy appearance of the cake and/or an increase in reconstitution time. In some cases, the collapse of a pharmaceutical product can be merely an aesthetic problem. In other cases, collapse can result in a suboptimal product. Generally, when a cake collapses, solvent becomes trapped within the cake and is not removed during secondary drying. This is generally undesirable because the additional solvent may reduce the stability of the final freeze-dried product. Meltback refers to a condition where some or all of the amorphous component becomes liquid. Thus, meltback is a severe form of collapse.

Although the collapse temperature is often equated with the glass transition temperature, they are not actually equivalent. The glass transition temperature is measured in a closed system of constant composition, whereas collapse is a dynamic process that can occur during the drying process. The Tg′ and the collapse temperature can differ by between 2° C. and 10° C.

Pressure

Another important parameter during the primary drying process is pressure. The pressure can affect the drying rate during primary drying as well as the state of the bulking agent (i.e., crystalline versus amorphous). Generally, the rate of sublimation from a frozen solid to vapor depends upon the difference in vapor pressure of the solid compared to the vapor pressure of the chamber. The pressure gradient is important because molecules migrate from the high-pressure crystalline component to the low-pressure chamber. Thus, the primary drying process is typically performed at a reduced pressure (i.e., under a vacuum). More typically, the chamber pressure is reduced to a pressure that is below the vapor pressure of the crystalline component. Because the vapor pressure of the crystalline component can vary depending on the formulation, the pressure at which the primary drying process is performed can vary, but is typically between about 40 mTorr and about 400 mTorr or between about 50 mTorr and about 200 mTorr.

Generally, at low pressures, the main form of heat transfer is conduction from the shelf through the bottoms of the product container. Since the product containers are typically glass and glass is an insulator, conduction is not very efficient and drying can be slow. Therefore, it may be desirable to improve the heat transfer mechanism by introducing an inert gas in to the drying chamber at a controlled rate. Examples of suitable inert gasses include nitrogen. The presence of the inert gas molecules facilitates heating of the walls of the container in addition to conduction through the bottom of the container, thereby increasing the amount of heat being supplied to the product per unit time. This enhances the drying rate, reduces the cycle time and reduces energy and labor costs associated with a lengthy process.

Additionally, because vapor pressure is related to temperature, the product temperature should be warmer than the cold trap temperature. Therefore, it is important that the temperature at which a product is freeze-dried is balanced between the temperature that maintains the frozen integrity of the product and the temperatures that increase the vapor pressure of the product.

Secondary Drying

The goal of the secondary drying process is to obtain a porous “freeze-dried formulation,” also referred to as a “cake” with a level of residual moisture that no longer supports biological growth and/or chemical reactions. In general, the secondary drying process removes the remaining unfrozen solvent trapped within (or adsorbed to) the amorphous solid matrix. However, many proteins require a solvent such as water to maintain proper secondary and tertiary structure. Therefore, it may not be desirable to remove all of the solvent from the cake. Generally, at the end of the secondary drying process, the freeze-dried formulation has a moisture content below about 8 wt %, below about 5 wt %, or typically between about 0 wt % to 3 wt %. It has been observed that moisture levels less than about 8% do not result in observable differences in purity or charge heterogeneity of the active agent, for example, when stored at a temperature of less than about 10° C., less than about 5° C., or between about 2° C. and about 5° C.

As used herein, the term “freeze-dried formulation” or “cake” refers to the dried formulation that remains after the solvent has been removed by the process of lyophilization. The freeze-dried formulation typically includes an amorphous solid matrix and a minor amount of unfrozen solvent. As used herein, the term amorphous solid matrix contains the active agent and excipients minus the solvent. It is worthwhile to note that the amorphous solid matrix can include both crystalline and amorphous excipients. As mentioned previously, an excipient, such as a bulking agent, may be present in a substantially crystalline or substantially amorphous form, or in both a crystalline an amorphous form.

Since there is very little mobile solvent in the formulation at the end of the primary drying stage, the shelf temperature may be increased during secondary drying without altering the structure of the resulting cake (i.e., causing melting). Additionally, the solvent remaining during secondary drying is typically more strongly bound to the amorphous solid matrix, and consequently may require more energy for its removal. Thus, during the secondary drying process, the shelf temperature is typically increased and the chamber pressure is decreased. Generally, the temperature of the secondary drying process may vary. Generally, a higher temperature results in a faster drying rate, although a temperature that is too high may result in collapse of the product. However, it is still important that the formulation remain frozen during the secondary drying process. Typically, a secondary drying process is performed at a temperature between about −20° C. and about 50° C., or between about 0° C. and about 40° C., or between about 10° C. and about 40° C. As water leaves the amorphous phase during secondary drying, Tg′ increases, which may allow the temperature to be increased further. Final temperature is a key factor in determining residual moisture in the dried cake.

Typically, the secondary drying process is performed under a vacuum, typically at about the same pressure as the primary drying process, typically between about 40 mTorr and about 400 mTorr or between about 50 mTorr and about 200 mTorr.

The Final Product—Freeze-Dried Formulation

In addition to obtaining a storage-stable formulation (i.e., one with appropriate residual moisture), another goal of the freeze-drying process is to obtain a freeze-dried formulation that retains the potency of the original liquid formulation upon reconstitution. The ability of the cake to dissolve rapidly and completely upon reconstitution is related to potency (filters used during administration may result in removal of incompletely reconstituted protein, resulting in reduced potency). Acceptable cake appearance is also important.

II. Formulations

As described above, lyophilization is a process in which a liquid formulation is subjected to a freeze-dry process to obtain a freeze-dried formulation. One goal of lyophilization is to retain the activity of the therapeutic agent while obtaining a pharmaceutically elegant end product.

The contents of a freeze-dried formulation may vary depending upon the active agent and the intended route of administration. The liquid formulation generally includes a solvent and solute. The solute typically includes an active agent and, optionally, one or more excipients. The resulting freeze-dried formulation typically includes an amorphous solid matrix and some residual unfrozen solvent. The amorphous solid matrix includes the active agent and, optionally, one or more excipients and, in some cases, residual solvent.

In general, any component in the formulation that is not the solvent or the active agent is referred to as an “excipient.” “Excipients” are included in a formulation for many reasons, although the primary function of many excipients is to provide a stable liquid environment for the active ingredient or to protect active agent during the freezing process. Some excipients may be used to achieve multiple effects in a formulation. For example, a disaccharide such as sucrose may act as a cryoprotectant, lyoprotectant, bulking agent and tonicity modifier. Behavior of an excipient may be different when in the presence of different excipients. For example, it may be desirable to include a crystalline bulking agent and a non-crystallizing lyoprotectant in a formulation such that the crystalline material provides a matrix, allowing primary drying to be conducted at high temperatures, while the non-crystallizing agent can serve as a lyoprotectant. Some combinations have a positive synergistic effect, while others have a negative synergistic effect. Positive synergy occurs when the sum of the effects of chemicals acting together is greater than the additive effects of the individual chemicals. Negative synergy occurs when the sum of effects of the mixture is less than that of the individual components of the mix. Examples of typical excipients are provided below.

Active Agent

As used herein, the term “pharmaceutical formulation” refers to both formulations that include active agents that are small molecule therapeutics and formulations that include a biopharmaceuticals as an active agent. As used herein, the term “small molecule therapeutics” refers to natural and synthetic substances that typically have a low molecular weight (i.e., less than about 1000 Daltons). Small molecules can be isolated from natural sources such as plants, fungi or microbes, or they can be synthesized by organic chemistry. Many conventional pharmaceuticals, such as aspirin, penicillin, and chemotherapeutics, are small molecules. The term “biopharmaceutical” refers to formulations containing active agents that generally have a high molecular weight (i.e., at least about 1000 Daltons). Examples of such “high molecular weight” active agents include carbohydrates and polypeptides.

The term “polypeptide” or “protein” as used herein can refer to both antibody and non-antibody proteins. Non-antibody proteins include, but are not limited to, proteins such as enzymes, receptors, and fragments thereof. The polypeptide may or may not glycosylated. The protein may or may not be fused to another protein. The term “antibodies” can include both monoclonal and polyclonal antibodies, antibody fragments, chimeric antibodies, human or humanized antibodies. Antibody fragments are known and include, but are not limited to, single chain antibodies, such as ScFv, Fab fragments, Fab′ fragments, etc. Antibodies tend to have a higher molecular weight than non-antibody proteins.

Solvent

As discussed previously, the lyophilization is the process by which solvent is removed from a liquid formulation. As used herein the term “solvent” refers to the liquid component of a formulation that is capable of dissolving or suspending one or more solutes. The term “solvent” can refer to a single solvent or a mixture of solvents. A commonly used solvent for pharmaceutical formulations is water for injection (WFI). Depending on the formulation or the freeze-drying process, it may be desirable to include one or more organic solvents in the liquid formulation.

Bulking Agents

The purpose of the bullring agent is to provide bulk to the formulation and enhance cake formation. Although bullring agents may improve cake structure, bulking agents may also reduce protein stability.

A variety of bullring agents are known. Common bullring agents include glycine and mannitol. Mannitol is a naturally occurring carbohydrate classified as a sugar alcohol or polyol. Glycine is a neutral amino acid. Some bullring agents, such as mannitol and glycine can form crystals during the freeze-drying process under some conditions.

As described above, a “crystalline solid” or “crystal” refers to a solid in which the constituent atoms, molecules or ions are packed in a regularly ordered, repeating pattern extending in all three spatial dimensions. An “amorphous solid” refers to a solid component that, unlike a crystalline solid, does not include a long-range order of the positions of the atoms, molecules or ions. It is worthwhile to note that a bulking agent can exist simultaneously in both a crystalline and amorphous state. For example, a substantially amorphous solid can include a minor amount (i.e., less than about 49 wt %) of crystalline solid. In one aspect of the invention, the substantially amorphous bulking agent includes less than about 25 wt %, 15 wt %, 10 wt %, 7 wt % or 5 wt % crystalline bulking agent.

The physical form of the bulking agent in the final lyophile may depend on formulation components, processing conditions and the concentration of the active agent. Components in the formulation such as lyoprotectants, including but not limited to, sucrose and trehalose may reduce the amount of bulking agent crystallization, e.g., mannitol crystallization. Process conditions may also affect the amount of crystalline or amorphous bulking agent. Furthermore, the combination of process conditions and the amount of active agent present in the formulation may affect the type and/or relative amount of the crystalline form of the bulking agent. For example, the process conditions in combination with the amount of active agent in the formulation may affect the amount of mannitol hemihydrate present in the final lyophile. In some instances, mannitol hemihydrate may not be desirable because it may lead to decreased stability of the active agent. As discussed previously, mannitol hemihydrate can retain water in its crystal lattice, which may not be removed during the lyophilization process. Therefore, if mannitol hemihydrate transforms to anhydrous mannitol during product storage, the previously entrapped water can be released. The release water will then be available for interaction with the other formulation components, including the active agent, which may lead to decreased stability of the active agent.

Stabilizing Agents

Stabilizing agents are typically added to a formulation to improve stability of the protein formulation, for example, by reducing denaturation, aggregation, deamidation and oxidation of the protein during the freeze-drying process as well as during storage. Examples of stabilizing agents include cryoprotectants and lyoprotectants. The term “cryoprotectant” refers to compounds that protect the protein against freezing. The term “lyoprotectant” refers to compounds that protect the protein during lyophilization.

Saccharides, including monosaccharides such as glucose, disaccharides such as sucrose (glucose+fructose), lactose (glucose+galactose), maltose (glucose+glucose), and trehalose (alpha-D-glucopyranosyl alpha-D-glucopyranoside), and polysaccharides such as dextran (polysaccharide containing glucose monomers) are commonly used stabilizing agents. Glucose, lactose and maltose are reducing sugars and can reduce proteins by means of the mailard reaction. Disaccharides such as sucrose, trehalose, maltose and lactose, and polysaccharides, such as dextran inert, are non-reducing sugars. A few hypothesis exist to explain the stabilizing effects of non-reducing sugars. The hydrogen-bonding theory postulates that the disaccharide stabilizer is able to form hydrogen bonds with protein (similar to the replaced water) which, in turn, prevents protein denaturation. This is also called the water replacement hypothesis. The preferential exclusion hypothesis postulates that the stabilizing agent is preferentially excluded from protein surface and destabilizes the unfolded state more than folded state. Thus, the thermodynamics of the system drives the protein towards folded (native) state. A final hypothesis is the vitrification hypothesis which postulates that disaccharides form sugar glasses of extremely high viscosity. The protein and water molecules are immobilized in the viscous glass, leading to extremely high activation energies required for any reactions to occur. It is believed that bulking agents, such as mannitol, are able to hydrogen bond with the protein and thus prevent denaturation

Surfactants

Surfactants, particularly nonionic surfactants, are often added to a formulation to reduce aggregation of the active agent during fermentation, purification, lyophilization, shipping, and/or storage. Aggregation of the active agent can compromise biological activity and has the potential to induce an immunological reaction when administered to a patient.

A number of surfactants are known. Common surfactants include, but are not limited to, polyoxyethylene sorbitan monolaurate (Tween™ 20, Tween™ 80), pluronic F-68, Triton™ X-100, and sodium dodecyl sulfate (SDS).

Nonionic surfactants are believed to protect active agents from damage by: 1) competing with active agents for adsorption sites on surfaces; 2) binding to hydrophobic regions on the surface of the active agent, thereby reducing intermolecular interactions; and/or 3) acting as a chemical chaperone, favoring refolding over aggregation by binding transiently with partially folded protein molecules and sterically hindering intermolecular interactions that result in aggregation.

Typically, surfactant is added to a liquid formulation at a concentration that is at or above the critical micelle concentration (CMC) for the surfactant. As used herein, the term “critical micelle concentration” refers to the concentration of an amphiphilic component, e.g., a surfactant, in solution at which the formation of aggregates in the solution is initiated. An amphiphilic molecule can arrange itself at the surface of the aqueous liquid such that the polar portion (hydrophilic portion) of the amphiphile interacts with the aqueous liquid and the non-polar portion (hydrophobic portion) of the amphiphile is held above the surface (either in the air or in a non-polar liquid). The presence of amphiphilic molecules on the surface disrupts the cohesive energy at the surface of the liquid and thus lowers the surface tension. Alternately, molecules can form aggregates in which the non-polar or hydrophobic portions are oriented within the cluster and the polar or hydrophilic portions are exposed to the aqueous or polar solvent. Such aggregates can show a variety of conformations, including, but not limited to micelles, round rods, and lamellar structures. The shape of the aggregates depends largely on the properties of the amphiphilic molecules. The proportion of molecules present at the surface of the solution or as aggregates within the solution depends on the concentration of the amphiphile. At low concentrations amphiphiles tend to favor arrangement on the surface. As the surface becomes crowded with amphiphiles more molecules arrange into aggregates. At some concentration the surface becomes crowded with amphiphile and additional amphiphile arrange into aggregates. The concentration at which these aggregates form is called the Critical Micelle Concentration (CMC). Methods for determining the CMC for a surfactant are known, for example, by examining the surface tension and/or conductivity of the solution. Generally, the CMC varies depending on the surfactant, in particular, the CMC may vary depending on the length of the hydrocarbon chain of the surfactant and the properties of the aqueous or non-polar solution. For example, the CMC for Tween™ 80 is generally between 0.01% and 0.02% wt/vol (in an aqueous solution such as water) whereas the CMC for Tween™ 20 is typically about 0.003% wt/vol (in an aqueous solution, such as water).

In one embodiment, the nonionic surfactant is included in the liquid formulation at or above the CMC for the surfactant. In another embodiment, the nonionic surfactant is added at in an amount less than the CMC of the surfactant, for example, at less than 10%, 20%, 30%, 40%, 50%, 60%, 70%, 80%, or 90% of the CMC of the surfactant. In one embodiment, the nonionic surfactant is added in an amount between 30% and 70%, 40% and 60% or 45% and 55% of the CMC of the surfactant. It may be desirable to reduce the amount of surfactant in a formulation because some surfactants, including Tween™ 20 and Tween™ 80, can contain a low level of residual peroxide which can potentially affect the stability of oxidation-sensitive active agents. Surfactants may also be susceptible to degradation during storage, wherein the oxidizing free radicals may compromise both the physical and chemical stability of the protein during long term storage. In one embodiment, a nonionic surfactant such as Tween™ 80, is included in a formulation at or above the CMC for the surfactant, for example, at a molar ratio concentration of nonionic surfactant:protein between about 10:1 and 30:1, more typically between 13:1 and 20:1. In another embodiment, a nonionic surfactant such as Tween™ 80 is included at a level below the CMC for the surfactant, for example, at a molar ratio concentration of nonionic surfactant:protein of between 1:1 and 10:1, for example, at a molar ratio concentration of nonionic surfactant:protein of less than 10:1, less than 8:1, less than 5:1 or less than 3:1. While not wishing to be bound by theory, it is believed that the surfactant can be included in a formulation at a level below the CMC for the surfactant when the protein included in the formulation binds the surfactant. While not wanting to be limited by theory, it is believed that the binding of the surfactant is driven by hydrophobic interactions between the surfactant and the protein. Examples of proteins that bind surfactant include, but are not limited to, growth hormone and human serum albumin, including fusion proteins containing growth hormone and/or human serum albumin. See, for example, Chou et al., “Effects of Tween™ 20 and Tween™ 80 on the Stability of Albutropin During Agitation,” J. Pharm. Sci. 94(6):1368-1381 (2005).

Isotonicity/Osmotic Pressure

When two solutions containing different particle concentrations are separated from each other by a semipermeable membrane, solvent will move across the membrane from the solution with the lower concentration to the solution with the higher concentration. The movement of the solvent will depend on the difference in the concentration of the particles and the nature of permeability of the membrane. This movement of solvent is termed osmosis and the pressure that would need to be exerted to halt its movement is called the osmotic pressure. It is important to realize that the osmotic pressure is determined by the total number of particles in solution, regardless of molecular nature. The total number of particles will thus depend on the degree of dissociation of solutes. For example, when added to water, sodium chloride dissociates into two ions per molecule, whereas sucrose does not dissociate. Thus, the osmotic pressure of a first solution containing a 1M concentration of sodium chloride will be twice the osmotic pressure as a second solution containing a 1M concentration of sucrose. Solutions that have the same osmotic pressure are called isotonic. A solution that has a lower osmotic pressure than another solution is called a hypotonic solution. A solution that has a higher osmotic pressure than another solution is called a hypertonic solution.

Osmolar concentration can be expressed in two ways: osmolality, which is expressed as mmol/kg of solvent and osmolarity, which is expressed as mmol/l of solution. Osmolality is a thermodynamically more precise expression because solution concentrations expressed on a weight basis are temperature independent while those based on volume will vary with temperature in a manner dependent on the thermal expansion of the solution.

Although the terms tonicity and osmolality are often used interchangeably, there is a clear distinction. Osmolality is a physical property dependent on the total number of solute particles present in a solution whereas tonicity is a physiological process dependent upon the selectively permeable characteristics of a membrane. For example, solutes that permeate cells freely have no effect on tonicity but increase the measured osmolality.

In normal humans the osmolality of body fluids is tightly regulated. Normal serum osmolality lies between 285 mOsm and 290 mOsm. Because movement of solvent from solutions having a low osmotic pressure to solutions having a high osmotic pressure can cause severe physiological problems, including cell dehydration (crenation) or expansion of the cell until it breaks open (lysis), osmotic pressure is an important consideration when preparing a pharmaceutical formulation, particularly a subcutaneous formulation. Thus, although the tonicity of the formulation may vary depending upon the stability requirements of the formulation or for the route of administration, in many instances it is important that the formulation, particularly formulations for subcutaneous administration, have approximately the same osmotic pressure (i.e., isotonic) as the cellular fluid (i.e., within approx. 50 mOsm). Generally, cellular fluid has an osmotic pressure between about 285 mOsm and about 290 mOsm. Therefore, a pharmaceutical formulation for subcutaneous administration should have an osmotic pressure between about 250 mOsm and about 350 mOsm, more preferably between about 275 mOsm and about 300 mOsm. In some instances, the tonicity modifier is added to the liquid formulation before freeze-drying. In other instances, the tonicity modifier is added along with the diluent during reconstitution of the freeze-dried formulation. Excipients such as mannitol, sucrose, glycine, glycerol and sodium chloride are good tonicity adjusters.

pH or Buffering Agents

Buffers are typically included in pharmaceutical formulations to maintain the pH of the formulation at a physiologically acceptable pH. The desirable pH for a formulation may also be affected by the active agent. For example, biopharmaceutical active agents have a higher activity within a range of pH. Generally, the pH of the formulation is maintained between about 5.0 and about 8.0, more typically between about 5.5 and about 7.5, or between about 6.0 and about 7.2. Typically the buffer is included in the liquid formulation at a concentration between about 5 mM to about 50 mM, or between about 10 mM and 25 mM.

Examples of suitable buffers include buffers derived from an acid such as phosphate, aconitic, citric, gluaric, malic, succinic and carbonic acid. Typically, the buffer is employed as an alkali or alkaline earth salt of one of these acids. Frequently the buffer is phosphate or citrate, often citrate, for example sodium citrate or citric acid. Other suitable buffers include Tris and histidine buffers.

III. Controlling Crystallization of the Bulking Agent

While it is beneficial to include a bulking agent in a formulation, for example, to increase the bulk of the final formulation, to increase the temperature of the primary drying process and/or to provide cake structure, inclusion of a bulking agent is not without disadvantages. For example, bulking agents may crystallize during the lyophilization process or during storage of the final product, resulting in the destabilization of the protein active agent, collapse or product variability, for example, by releasing water associated with the amorphous phase. In some cases it may be desirable to keep the bulking agent in a substantially amorphous state throughout lyophilization and storage, rather than allowing the bulking agent to crystallize. While not intending to be bound by theory, it is believed that maintaining the bulking agent in a substantially amorphous state (i.e., less than 25 wt %, 15 wt %, 10 wt %, 7 wt % or 5 wt % crystalline) may enhance protein stability. Alternately, in other formulations, it may be desirable to generate a crystalline form of bulking agent and retain the crystalline form throughout lyophilization and storage. While not intending to be bound by theory, it is believed that maintaining the bulking agent in a substantially crystalline state (i.e., greater than 75 wt %, 85 wt %, 90 wt %, 93 wt %, or 95 wt % crystalline) may facilitate reconstitution of the final drug product.

It is worthwhile to note that crystallization is influenced by many factors, including, but not limited to formulation variables (e.g., concentration of active agent), processing conditions (e.g., freezing rates), as well as the presence of a non-crystallizing solute. In one aspect of the invention, a formulation is provided that includes a protein active agent and at least a bulking agent in which the bulking agent is maintained in a substantially amorphous state (i.e., in which the solid bulking agent is less than 25 wt %, 15 wt %, 10 wt %, 7 wt % or 5 wt % crystalline) during the freeze-drying process and/or storage. In another aspect of the invention, a formulation is provided that includes a protein active agent and at least a bulking agent in which the bulling agent is maintained in a substantially crystalline state (i.e., in which the solid bulling agent is greater than 75 wt %, 85 wt %, 90 wt %, 93 wt %, or 95 wt % crystalline) during the freeze-drying process and/or storage.

The inventors have found that crystallization of bulling agents such as mannitol can be influenced by both the bulking agent to saccharide stabilizing agent ratio and/or by the concentration of the protein active agent in the formulation. In one embodiment, the crystallization of the bulking agent is controlled by decreasing the weight ratio of bulking agent to saccharide stabilizing agent plus protein active agent in the formulation. Although not wishing to be bound by theory, the inventors believe that decreasing the ratio of bulking agent to other amorphous components in the formulation may interfere with the ability of the bulking agent to crystallize. Table 1 provides a summary of known liquid formulations that are used to generate freeze-dried formulations. In one embodiment, the saccharide is a disaccharide, such as sucrose and the bulking agent is mannitol.

TABLE 1

| | | [P]* | [M]* | [M] | [S]* | [S] | M:S | M:S | M:(S + P) | M:(S + P) |
|---|---|---|---|---|---|---|---|---|---|---|
| Company | Product | (mg) | (mg) | (mM) | (mg) | (mM) | (wt. ratio) | (molar ratio) | (wt. ratio) | (molar ratio) |
| Genentech | Cleland et al.** | 5 | 7.3 | 40 | 6.8 | 20 | 1.1:1 | 2:1 | 0.61:1 | 2:1 |
| Berlex | Sargromostim⁺ | 0.25 | 40⁺⁺ | 222 | 10 | 29 | 4:1 | 7.7:1 | 3.9:1 | 7.7:1 |
| Novartis | Simulect⁺ | 10 | 40⁺⁺ | 222 | 10 | 29 | 4:1 | 7.7:1 | 2:1 | 7.7:1 |
| Amgen | Enbrel⁺ | 25 | 40⁺⁺ | 222 | 10 | 29 | 4:1 | 7.7:1 | 1.14:1 | 7.7:1 |

*P = protein; M = mannitol; S = sucrose
**J. Pharm. Sci. 90(3): 310-321 (2001)
⁺Obtained from Physicians Desk Reference
⁺⁺The physical state of mannitol (i.e., whether crystalline or amorphous) is not provided

Bulking Agent

As discussed above, the term “bulking agent” refers to components of a formulation that provide bulk. In one embodiment of the invention, the bulking agent of a freeze-dried formulation is maintained in a substantially amorphous state that includes less than about 25wt % crystalline bulking agent, or less than about 15 wt %, lOwt %, 7 wt % or 5 wt % crystalline bulling agent. A variety of bulking agents are known, including, but not limited to glycine and mannitol. In one embodiment, the bulking agent is mannitol.

Saccharide Stabilizing Agent

As discussed above, stabilizing agents are typically added to a formulation to improve stability of the protein formulation. Although an increase in the concentration or amount of stabilizing agent may increase the stability of the protein formulation, the concentration or amount of stabilizing agent may be limited by practical considerations, such as the osmolality of the final dosage form. According to one embodiment, the saccharide stabilizing agent is a disaccharide. In another embodiment, the disaccharide is sucrose. In yet another embodiment, the ratio of bulking agent to saccharide stabilizing agent is between about 5:1 and about 0.2:1, or between about 3:1 and about 0.45:1.

Protein Active Agent

The concentration of protein active agent in a pharmaceutical formulation is influenced by the desired properties of the clinical product. For example, the efficacy of the product may be affected by the protein concentration. The desired protein concentration may also be affected by the route of administration. For example, a higher concentration of protein active agent is generally desirable for subcutaneous administration when compared to intravenous administration.

The inventors have found that the concentration of protein in a biopharmaceutical formulation can affect crystallization of a bulking agent, such as mannitol. In general, as the protein concentration in a liquid formulation is increased, the crystallization of mannitol during lyophilization and/or storage decreases. While not wishing to be bound by theory, it is believed that the complex structure of proteins, including numerous functional groups that can react non-specifically with the formulation components can influence the crystallization behavior of a bulking agent, such as mannitol. It is believed that the presence of protein may even inhibit crystallization of a bulking agent during lyophilization such that crystallization is incomplete in the final lyophilized product. However, the final protein concentration may be limited by the solubility of the protein upon reconstitution. According to one aspect of the invention, the biopharmaceutical formulation of the invention includes one or more proteins at a concentration of at least about 0.1 mg/ml. Typically, the protein active agent is included in the liquid formulation at a concentration between about 0.1 mg/ml and 100 mg/ml.

Process Parameters

The physical state of the bulking agent (i.e., whether it exists as an amorphous solid or a crystalline solid) at the end of the freeze-drying process can be affected by the processing parameters used during the freeze-drying cycle (cooling rate, annealing time and temperature, primary and secondary drying conditions). According to one embodiment of the invention, the freeze-drying process is carried out under conditions sufficient to maintain the bulking agent in a substantially amorphous state (i.e., wherein less than about 25 wt % of the bulking agent present in the formulation is in a crystalline state). According to another embodiment, the freeze-drying process is carried out under conditions sufficient to maintain the bulking agent in a substantially crystalline state (i.e., wherein greater than about 75 wt % of the bulking agent present in the formulation is in a crystalline state).

IV. Modes of Administration

The freeze-dried formulation of the invention is suitable for parenteral administration, including intravenous, subcutaneous and intramuscular administration.

Having generally described the invention, the same will be more readily understood by reference to the following examples, which are provided by way of illustration and are not intended as limiting. The entire disclosure of each document cited (including patents, patent applications, journal articles, abstracts, laboratory manuals, books, or other disclosures) in the disclosure is hereby incorporated herein by reference.

### WORKING EXAMPLES

### Example 1

Differential scanning calorimetry (DSC) and X-ray powder diffractometry (XRD) were used to characterize the crystallization of mannitol is various formulations. In the DSC, the solutions were cooled from room temperature to −70° C. at 20° C./minute, held for 40 minutes, and heated to RT at 5° C./minute. The annealing temperature ranged from −49° C. to −37° C., while the annealing time ranged from 15 to 480 minutes. The diffraction patterns were obtained in a wide angle X-ray powder diffractometer (CuKα radiation; 45 kV×40 mA). The sample was subjected to a controlled temperature program ranging from −70° C. to 25° C. and exposed to CuKα radiation in the continuous mode at chopper increments of 0.05° 2θ. The angular range was 5 to 40° 2θ, the step size was 0.05° 2θ and the dwell time was 1 sec.

### Example 1a

### The Effect of Mannitol to Sucrose Ratio on Tg′

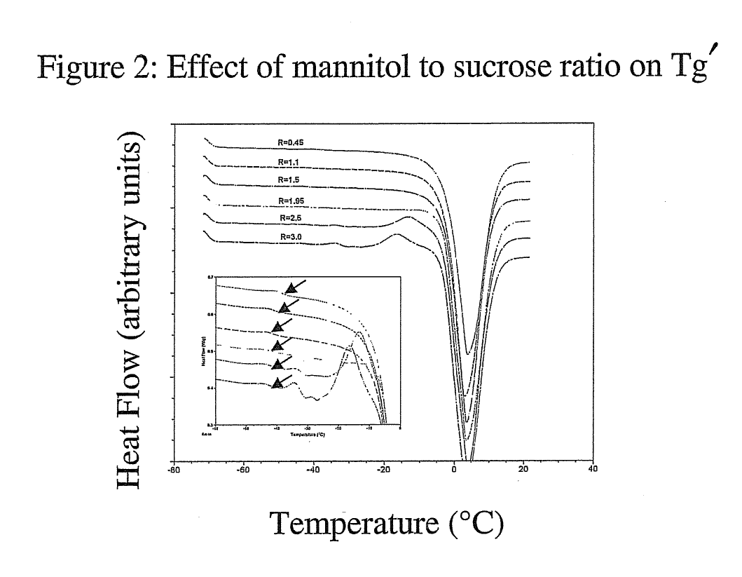

FIG. 2 shows the effect of the mannitol to sucrose ratio (R) on Tg′ (no protein). As shown in the figure, the Tg′ of a formulation containing mannitol and sucrose is dependant on the mannitol to sucrose ratio (R). As the ratio of mannitol to sucrose (R) was increased, the Tg′ decreased until the mannitol to sucrose reached a ratio of 1.5:1. At mannitol to sucrose ratios (R) greater than 1.5:1, Tg′ was relatively constant. At mannitol to sucrose ratios (R) greater than 2.5:1, mannitol crystallization was evident.

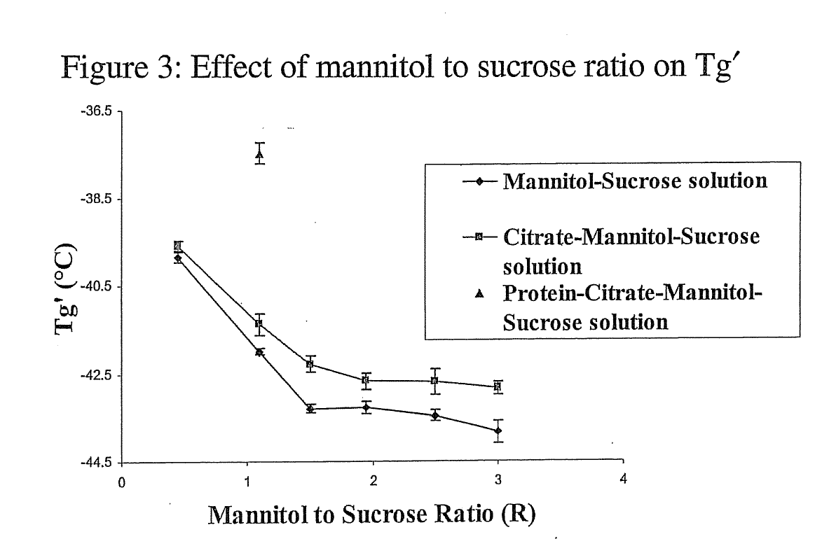

FIG. 3 shows the effect of the mannitol to sucrose ratio (R) on Tg′ in the presence of 20 mg/ml protein. An increase in the mannitol to sucrose ratio (R) in the presence of protein caused a similar decrease in Tg′ as that of mannitol and sucrose without protein (See FIG. 2). However, the addition of protein (20 mg/ml) raised Tg′ by approximately 4° C. This effect on Tg′ was evident for mannitol to sucrose ratios (R) in the range of 0.5:1 to about 1.5:1 where Tg′ stabilized. At mannitol to sucrose ratios (R) greater than 1.5:1, up to 3:1, the increase in the mannitol to sucrose ratio (R) had a less dramatic effect on Tg′. The presence of citrate buffer in the mannitol-sucrose solution also slightly increased Tg′.

### Example 1b

### The Effect of Protein Concentration on Tg′

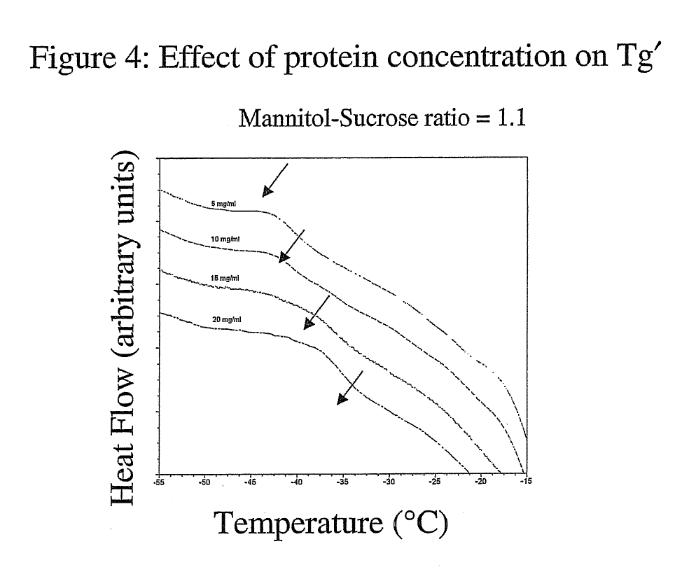

FIG. 4 shows the effect of increased protein concentrations on Tg′ in solutions containing mannitol and sucrose. The ratio of mannitol to sucrose (R) was 1.1. As the protein concentration was increased from 5 mg/ml to 20 mg/ml, the Tg′ also increased. This suggests that primary drying may be performed at progressively higher temperatures.

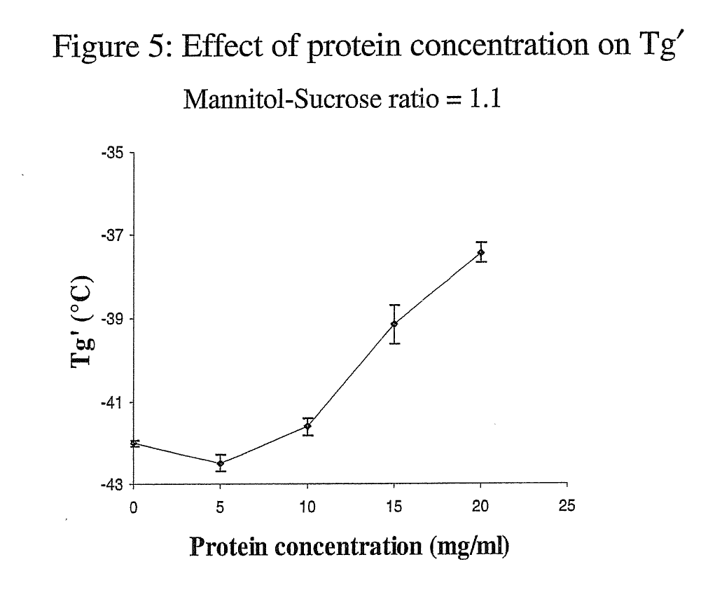

FIG. 5 shows the effect of increased protein concentrations on Tg′ for solutions containing mannitol and sucrose at a ratio (R) of 1.1. As the protein concentration increased, the Tg′ increased, suggesting that primary drying may be performed at progressively higher temperatures.

### Example 1c

### The Effect of the Annealing Temperature and Time

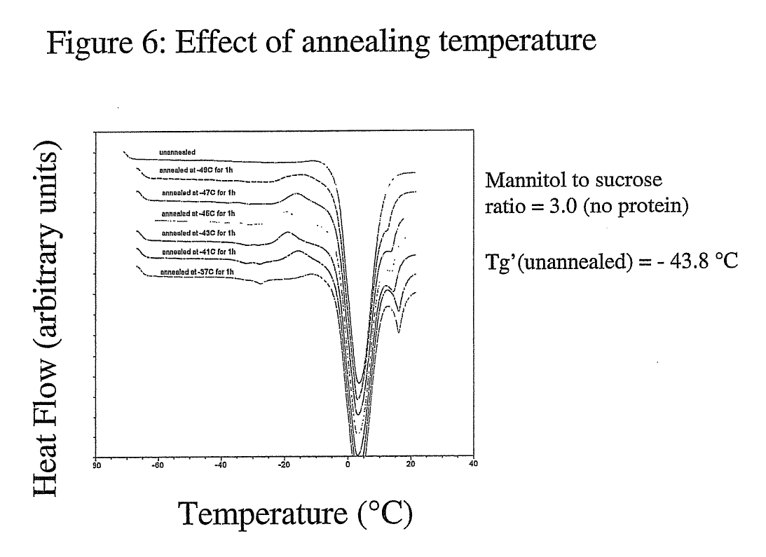

FIG. 6 shows the effect of the annealing temperature (both above and below Tg′) on a solution containing mannitol and sucrose at a ratio (R) of 3.0, in the absence of protein. As shown in FIG. 6, annealing (at both above and below Tg′) increased mannitol crystallization. Annealing at −45° C. resulted in the lowest crystallization onset temperature and the maximum enthalpy of crystallization, suggesting maximum nucleation at this temperature. Tg′ was −43.8° C. These results suggest that mannitol crystallization may allow primary drying to be carried out at an elevated temperature.

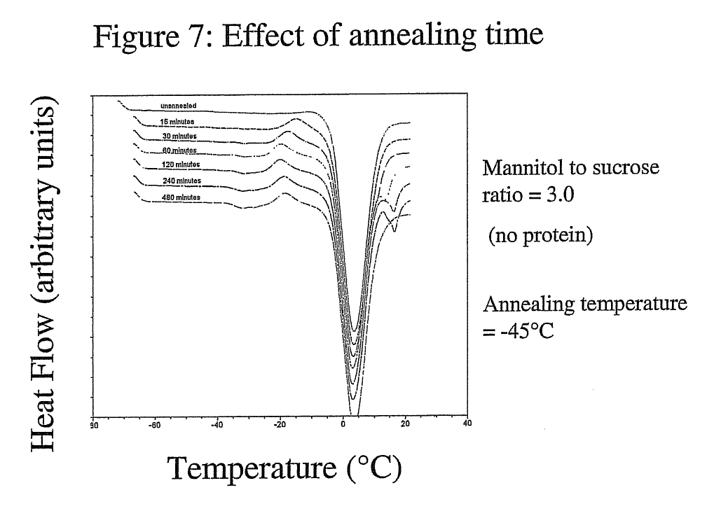

FIG. 7 shows the effect of the annealing time on a mannitol-sucrose solution at a ratio (R) of 3.0, no protein, at an annealing temperature of −45° C. As the annealing time was increased from 15 minutes to 480 minutes, the mannitol crystallization onset temperature decreased. Additionally, as the annealing time increased, the enthalpy of mannitol crystallization also increased.

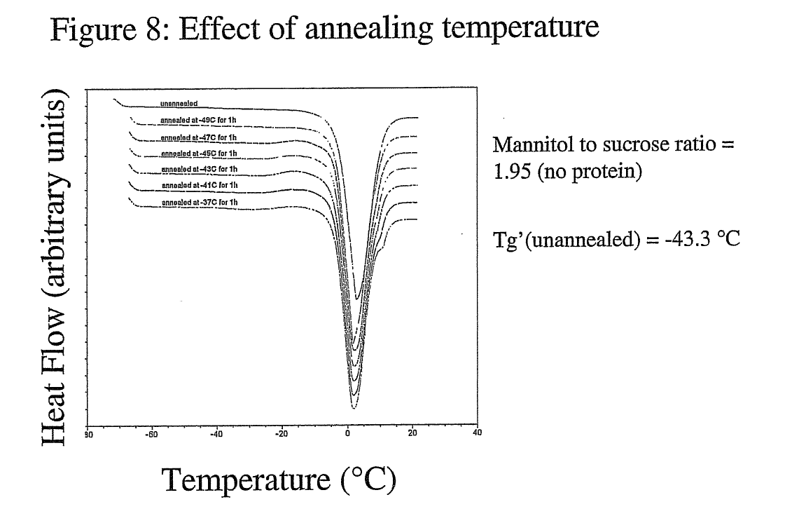

FIG. 8 shows the effect of the annealing temperature on a mannitol to sucrose solution at a ratio (R) of 1.95, no protein. When compared to FIG. 6, FIG. 8 demonstrates that increasing the amount of sucrose relative to mannitol appears to inhibit mannitol crystallization when R=1.95. Annealing facilitated mannitol crystallization only when annealed at temperatures greater than or equal to −47° C. There is no evidence on mannitol crystallization when annealed at −49° C.

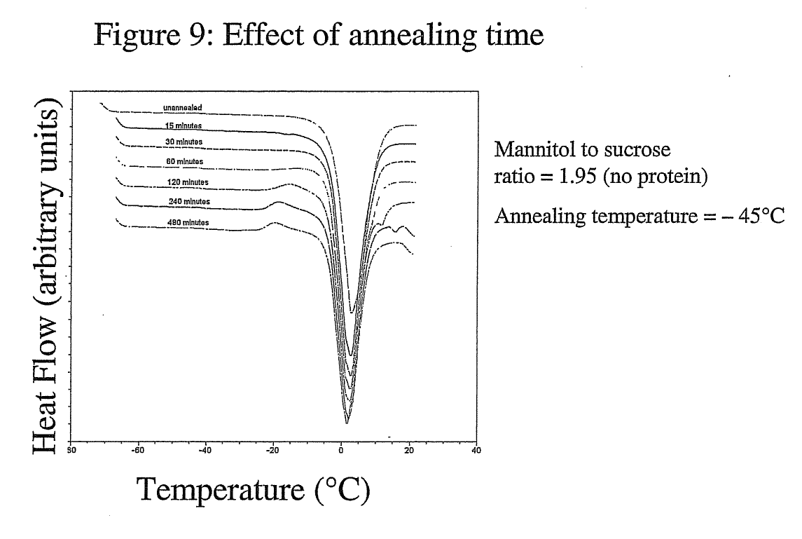

FIG. 9 shows the effect of the annealing time on a mannitol to sucrose solution at a ratio (R) of 1.95, no protein, at an annealing temperature of −45° C. As compared to FIG. 7, increasing the amount of sucrose relative to mannitol appears to inhibit mannitol crystallization. At a mannitol to sucrose ratio (R) of 1.95, mannitol crystallized only after annealing for 2 hours.

### Example 1d

### Real Time Monitoring During Freeze-Drying by in situ XRD—

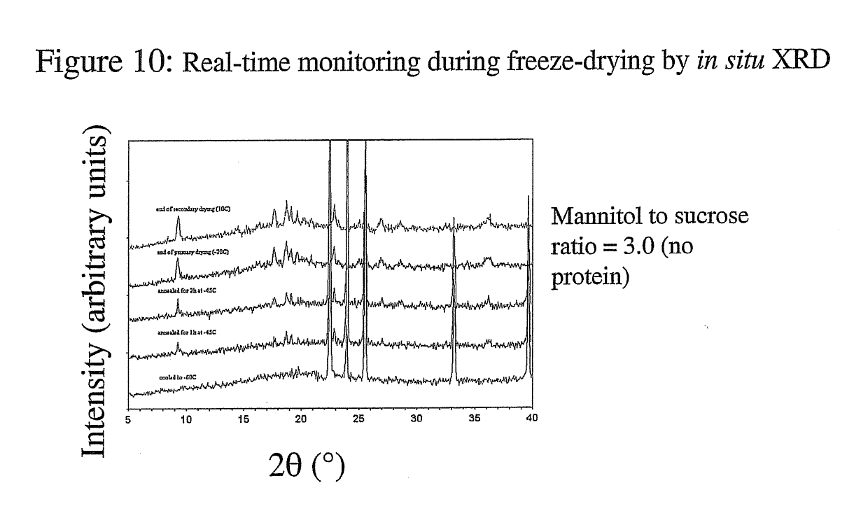

FIG. 10 shows real time monitoring of mannitol crystallization during freeze-drying by in situ XRD for a solution containing a mannitol to sucrose ratio (R) of 3.0, no protein. The presence of crystalline mannitol is shown by the characteristic peak of mannitol hydrate at 19.0° 2θ. FIG. 10 shows that mannitol crystallized during annealing for 1 hour at −45° C. (Tg′=−44° C.). Crystalline mannitol formed readily upon freeze drying in the absence of protein.

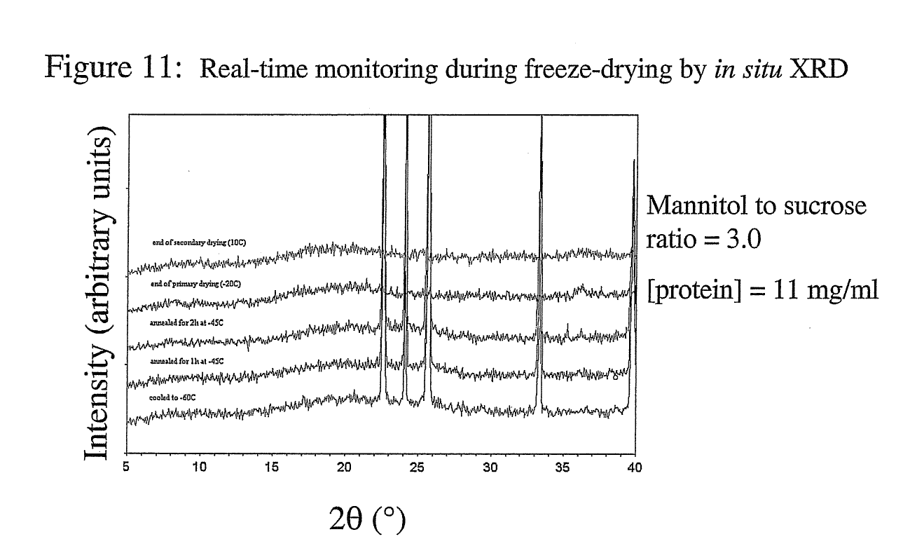

FIG. 11 shows real time monitoring by in situ XRD of mannitol crystallization during freeze-drying of a solution containing a mannitol to sucrose ratio (R) of 3.0, with 11 mg/ml protein. In contrast to FIG. 10, in the presence of protein, annealing at −45° C., for 1 to 2 hours did not result in mannitol crystallization. Additionally, no mannitol crystallization was observed during primary or secondary drying.

### Example 2

### The Effect of the Active Agent on the Physical State of Mannitol in a Lyophilized Monoclonal Antibody Formulation

A human monoclonal antibody was the active agent and mannitol and sucrose were the bulking agent and the lyoprotectant, respectively. The thermal behavior of frozen mannitol-sucrose solutions during and after annealing, in the absence and presence of the protein, were characterized using low temperature X-ray powder diffractometry (XRD) and differential scanning calorimetry (DSC). The influence of the protein on the crystallization behavior of mannitol during various stages of freeze-drying was also evaluated.

In the liquid formulation, the protein concentration ranged between 10 and 50 mg/ml, which is high compared to many other protein formulations (see, e.g., Kreilgaard et al. (1998) “Effects of additives on the stability of recombinant human factor XIII during freeze-drying and storage in the dried solid.” Archives of Biochemistry and Biophysics 360: 121-134 and Lam et al. (2001) “Encapsulation and stabilization of nerve growth factor into poly(lactic-co-glycolic) acid microspheres.” Journal of Pharmaceutical Sciences 90: 1356-1365).

The following was evaluated: (i) the effect of the model protein on the behavior of the mannitol-sucrose frozen solutions under subambient conditions, (ii) the effect of protein on the crystallization behavior of mannitol, and (iii) the effect of thermal treatment (annealing) on mannitol crystallization. The thermal behavior of frozen mannitol-sucrose solutions during and after annealing, in the absence and presence of the protein, were characterized using low temperature X-ray powder diffractometry (XRD) and differential scanning calorimetry (DSC). The influence of the protein on the crystallization behavior of mannitol during various stages of freeze-drying was also evaluated.

Materials

D-Mannitol (C₆H₁₄O₆, Sigma, St. Louis, Mo.), sucrose (C₁₂H₂₂O₁₁, Aldrich, Milwaukee, Wis.), and citric acid (C₆H₈O₇, Sigma, St. Louis, Mo.) were used as received. The studies were divided into three groups, with a progressive increase in the number of components. The aqueous solutions contained (1) different weight ratios of mannitol to sucrose; (2) the same weight ratio of mannitol to sucrose in the presence of citrate buffer; (3) mannitol, sucrose, citrate buffer and the protein. The sucrose and mannitol concentrations were in the range of 2-5% (w/v). The mannitol to sucrose weight ratios (R) were 0.45, 1.10, 1.50, 1.95, 2.50 and 3.00. The detailed solution compositions are provided in Table 2. The citrate buffer concentration was 10 mM and the solution pH was 6.5. When the human monoclonal antibody was added (hereafter referred as ‘protein’), its concentration ranged between 10 and 50 mg/ml. All solutions were subjected to membrane filtration (0.45 μm) except the protein solution.

Methods

Differential Scanning Calorimetry (DSC)

A differential scanning calorimeter (MDSC, Model 2920, TA Instruments, New Castle, Del.) with a refrigerated cooling accessory was used. The DSC cell was calibrated using mercury and distilled water. About 12-14 mg of the sample solution was weighed in an aluminum pan, sealed hermetically, cooled from room temperature to −70° C. at 20° C./min and maintained at −70° C. for 30 minutes. The frozen solutions were then heated at 5° C./min to room temperature. Only the DSC heating curves were recorded. When there was a thermal treatment (annealing) step, the frozen solutions were annealed at temperatures ranging from −25 to −45° C. for time-periods of 15 to 480 minutes.

X-Ray Powder Diffractometry (XRD)

An X-ray powder diffractometer (Model XDS 2000, Scintag) with a variable temperature stage (Micristar, Model 828D, R.G. Hansen & Associates, Santa Barbara, Calif.; working temperature range −190 to 300° C.) was used. A vacuum pump was attached to the temperature stage of the XRD, which allowed the entire lyophilization process to be simulated in the sample chamber of the XRD. An accurately controlled aliquot of sample solution (−100 mg) was filled into a copper holder and cooled at a constant predetermined rate from room temperature to −70° at 10° C./min. The samples were then normally held for 30 minutes and heated to the annealing or primary drying temperature at 5° C./min.

Results and Discussion

The excipient concentrations had a pronounced effect on the Tg′ (glass transition temperature of maximally freeze-concentrated amorphous phase). At fixed excipient compositions, the protein had little effect on the Tg′ if the protein concentration was ≦20 mg/ml. However, as the protein concentrations were increased, there was a marked increase in Tg′. In situ XRD provided evidence of the concentration-dependent inhibitory effect of the protein on mannitol crystallization. Annealing facilitated mannitol nucleation as well as crystal growth.

Characterization of Frozen Aqueous Mannitol-Sucrose Solutions

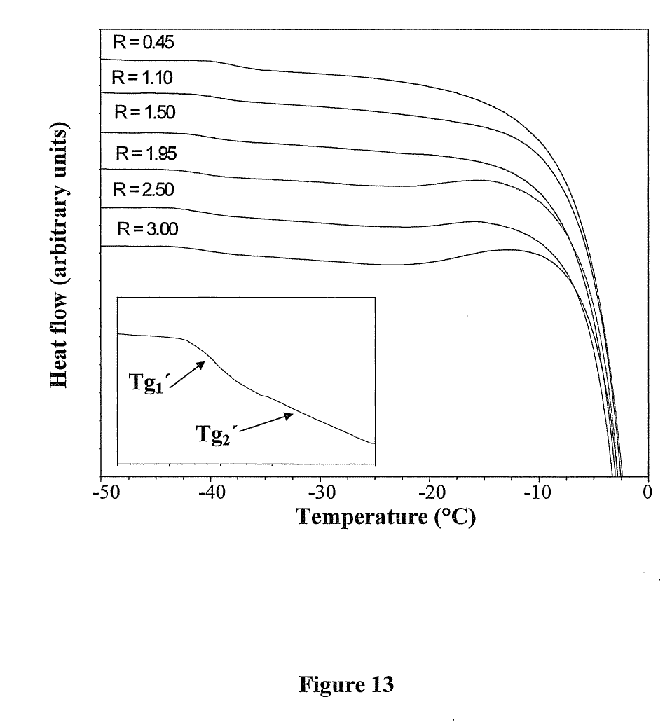

Initial studies focused on the thermal events during the cooling and heating of aqueous solutions containing only mannitol and sucrose. It has been reported, and confirmed in the current investigation, that at cooling rates ≧20° C./min, mannitol crystallization is inhibited (see, e.g., Pyne et al. (2002) “Crystallization of Mannitol below Tg′ during Freeze-Drying in Binary and Ternary Aqueous Systems.” Pharmaceutical Research 19: 901-908). On the other hand, when the frozen aqueous solution was heated to room temperature at 5° C./min, several thermal events were observed (FIG. 13): (i) glass transition with onset between −40 and −44° C. (Tg′₁), (ii) a possible second glass transition with onset at approximately −27° C. (Tg′₂), (iii) depending on the mannitol to sucrose weight ratio (R), an exotheim attributable to solute crystallization when R≧1.95; and (iv) an endotherm due to eutectic melting of mannitol and ice. The origin of multiple glass transitions is not fully understood and is a subject of debate (see, e.g., Chang et al. 1999. The origin of multiple glass transitions in frozen aqueous solutions. Proceedings of the NATAS Annual Conference on Thermal Analysis and Applications 27th: 624-628).

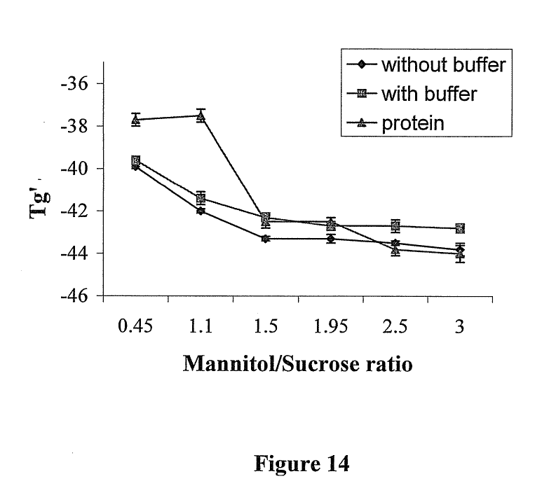

Tg′ (glass transition temperature of maximally freeze-concentrated amorphous phase) is one of the important thermophysical parameters in the design of the lyophilization cycle since it is very close to the collapse temperature (see, e.g., Tang et al. 2004. Design of Freeze-Drying Processes for Pharmaceuticals: Practical Advice. Pharmaceutical Research 21: 191-200). Tg′ normally forms the basis for selection of the primary drying temperature. Therefore, our studies initially focused on the effect of the mannitol to sucrose weight ratio (R) on the Tg′. As shown in FIG. 14, as R increased from 0.48 to 1.5, the Tg′ decreased from −39 to −43° C. At R value ≧1.5, the Tg′ reached a plateau (−44.6° C.). Interestingly, the Tg′ of the mixture is lower than the Tg′ of the individual components, i.e. mannitol (−30° C.) and sucrose (−35° C.). It has been speculated that the unfrozen water content increased as the mannitol to sucrose ratio increased (see, e.g., Lueckel et al. (1998) “Formulations of sugars with amino acids or mannitol-influence of concentration ratio on the properties of the freeze-concentrate and the lyophilizate.” Pharmaceutical Development and Technology 3: 325-336).

The effect of buffer on the Tg′ was also examined by replacing water with 10 mM aqueous citrate buffer (pH=6.5). A similar trend was observed although in the presence of the citrate buffer the Tg′ increased by about 1° C. over the entire range. The Tg′ value at R=1.95 (˜−42° C.) is in reasonable agreement with previously reported value of ˜−41° C. (R=2) (see, e.g., Martini et al. (1997) “Use of Subambient Differential Scanning Calorimetry to Monitor the Frozen-State Behavior of Blends of Excipients for Freeze-Drying.” PDA Journal of Pharmaceutical Science & Technology 51: 62-67).

The effect of protein, at a concentration of 20 mg/ml, was investigated. As shown in FIG. 14, the Tg′ stayed almost unchanged when R increased from 0.45 to 1.10, followed by a sharp drop at R=1.50. The Tg′ continued to decrease until the R reached 2.50. It is well known that Tg′ influences the selection of the primary drying temperature. However, in the mannitol-sucrose formulation, although the Tg′ is low (˜−42° C.), the primary drying can still be conducted at a relatively high temperature (˜−10° C.). It is postulated that crystalline mannitol supports the weight of the lyophile and prevents macroscopic collapse. The primary drying temperature thus appears to be dependent on the fraction of crystalline phase in the formulation, as has been demonstrated in recent examples (see, e.g., Johnson et al. (2002) “Mannitol-sucrose mixtures-versatile formulations for protein lyophilization. Journal of Pharmaceutical Sciences 91: 914-922 and Chatterjee et al. In press. Partially Crystalline Systems in Lyophilization: II. Withstanding Collapse at High Primary Drying Temperatures and Impact on Protein Activity Recovery. Journal of Pharmaceutical Sciences).

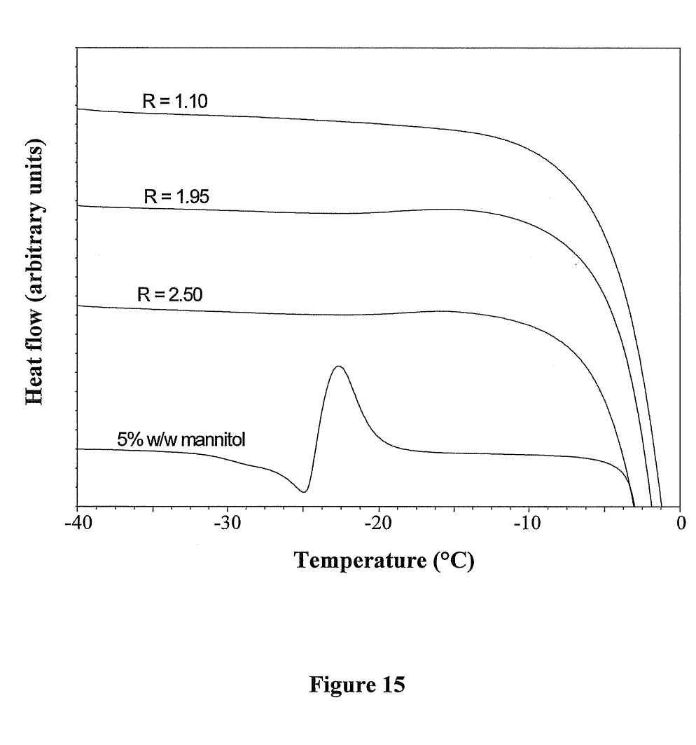

The inhibitory effect of sucrose on mannitol crystallization is also a subject of study. It is well known that sucrose, a noncrystallizing solute, prevents mannitol crystallization (see, e.g., Martini et al. (1997) “Use of Subambient Differential Scanning Calorimetry to Monitor the Frozen-State Behavior of Blends of Excipients for Freeze-Drying.” PDA Journal of Pharmaceutical Science & Technology 51: 62-67). The extent of inhibition depends on the concentration ratio of mannitol to sucrose. As shown in FIG. 15, when R is <1.5, no crystallization exotherm was observed in the DSC profiles. A very small exotherm appeared just before the eutectic melting endotherm, at R>1.95. A control experiment was conducted, using 5% mannitol without sucrose. A much sharper exotherm was seen with an onset temperature at ˜−25.0° C. Mannitol only crystallizes when R is >1.95, which is a good agreement with previous studies (see, e.g., Johnson et al. (2002) “Mannitol-sucrose mixtures-versatile formulations for protein lyophilization.” Journal of Pharmaceutical Sciences 91: 914-922).

Effect of Protein Concentration on Tg′ and Mannitol Crystallization

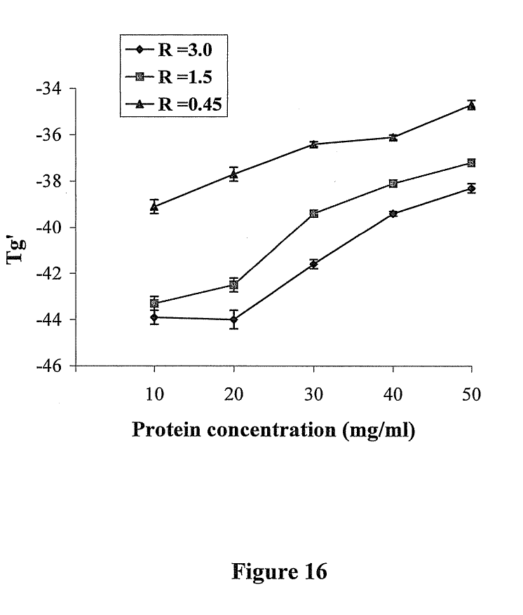

From FIG. 14, it is evident that in the presence of the protein, at a concentration of 20 mg/ml, influences the Tg′ at R<1.5. The effect of protein concentration on the Tg′ was determined, as a function of protein concentrations at R values of 0.45, 1.5 and 3.0. As shown in FIG. 16, at R=0.45, Tg′ increased 4.4° C., from −39.1 to −34.7° C. as the protein concentration was increased from 10 to 50 mg/ml. At higher R values (1.5 and 3.0), at low protein concentrations (10 and 20 mg/ml), there appeared to be little or no effect on the Tg′. However, at higher protein concentrations, there was a marked increase in Tg′ as a function of protein concentration. At R values of 1.5 and 3.0, if the protein concentration is high (>20 mg/mil), the Tg′ is sensitive to protein concentration. This can be very important in the design of lyophilization cycles if formulations with different strengths of active agents are contemplated.

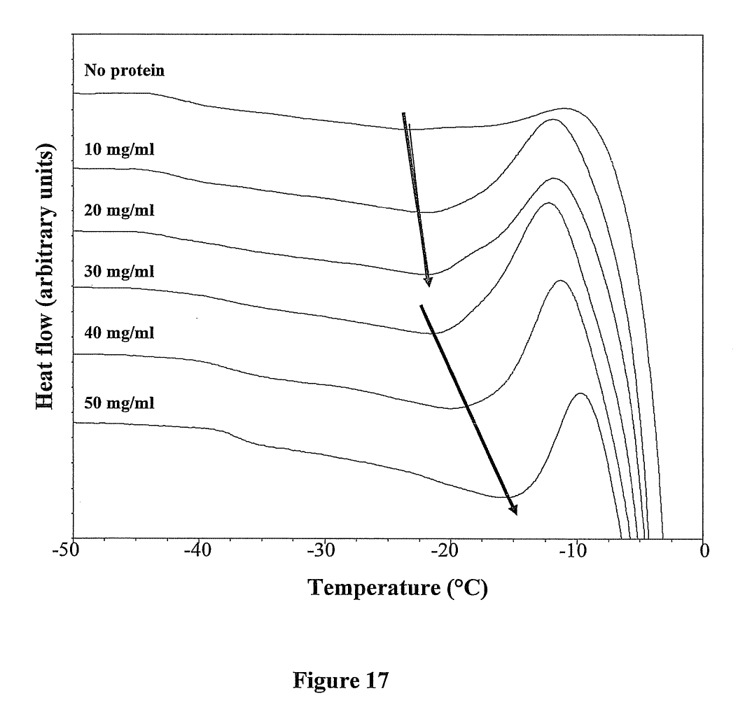

The inhibitory effect of protein concentration on mannitol crystallization was also investigated. Solutions (R=3.0) were chosen because mannitol crystallization was evident in such system. As shown in FIG. 17, when protein concentrations increased from zero to 20 mg/ml, the crystallization onset temperature of mannitol shifted slightly to higher temperature. As the protein concentration increased from 30 to 50 mg/ml, the onset temperature increased from −21 to −15° C. The crystallization onset temperature shifts to higher temperature is a clear indication of the inhibitory effect of the protein on mannitol crystallization.

Effect of Annealing

Nucleation is a prerequisite of crystallization (see, e.g., Searles et al. 2001. Annealing to optimize the primary drying rate, reduce freezing-induced drying rate heterogeneity, and determine Tg′ in pharmaceutical lyophilization. Journal of Pharmaceutical Sciences 90: 872-887). Previous studies of a mannitol-trehalose system revealed that sub-Tg′ annealing facilitated ice crystallization and mannitol nucleation (see, e.g., Pyne et al. 2002. Crystallization of Mannitol below Tg′ during Freeze-Drying in Binary and Ternary Aqueous Systems. Pharmaceutical Research 19: 901-908).

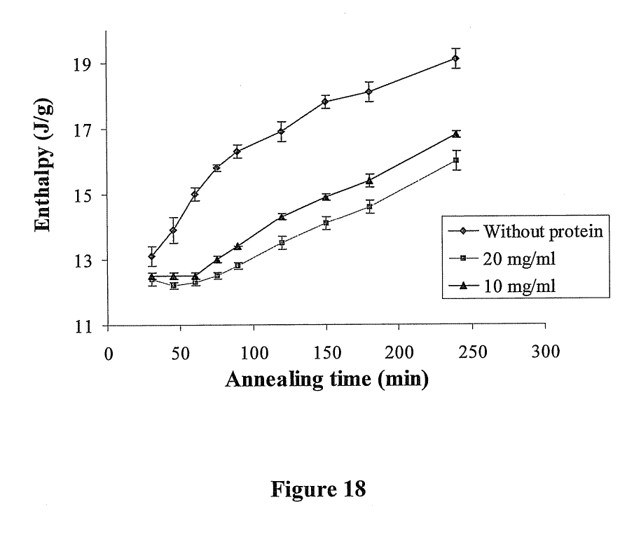

The effect of sub-Tg′ annealing in the absence and the presence of the protein was investigated. The solution at R=3.0 was studied in detail since it had the most pronounced crystallization event in the DSC profile. The annealing temperature (Ta) was −45° C., one degree below the Tg′. Since the mannitol-sucrose solution and the mannitol-sucrose-protein solution have almost the same Tg′, the difference between the annealing temperature and the glass transition temperature (Ta−Tg′) was about the same. FIG. 18 shows the plot of enthalpy of crystallization versus the annealing time when the sample was annealed at −45° C. For unannealed solutions, the crystallization onset was delayed so that the crystallization exotherm overlapped with the huge eutectic melting endotherm, thus making the accurate measurement of crystallization enthalpy difficult. For annealed samples, the annealing led to nucleation, which resulting in crystallization at lower temperatures. This separated the crystallization exotherm from the eutectic melting endotherm enabling the accurate measurement of enthalpy.

FIG. 18 shows that the enthalpy of crystallization increased with the annealing time. In the absence of protein, the enthalpy increased from 13.1 to 19.1 J/g when the annealing time was increased to 240 minutes from 30 minutes. With the addition of protein (10 mg/ml), the enthalpy increased from 12.5 to 16.8 J/g in the same time period. As the protein concentration increased to 20 mg/ml, the enthalpy increased from 12.4 J/g to 16.0 J/g. The increase in the enthalpy of crystallization with annealing time was more pronounced in the absence of protein. It can be inferred that the protein exhibits a concentration dependent inhibition of mannitol crystallization. In the absence of the protein, mannitol crystallization was initiated almost immediately. On the other hand, there was a lag time of 30 and 60 minutes at protein concentrations of 10 and 20 mg/ml, respectively.

Effect of Annealing on Mannitol Crystallization

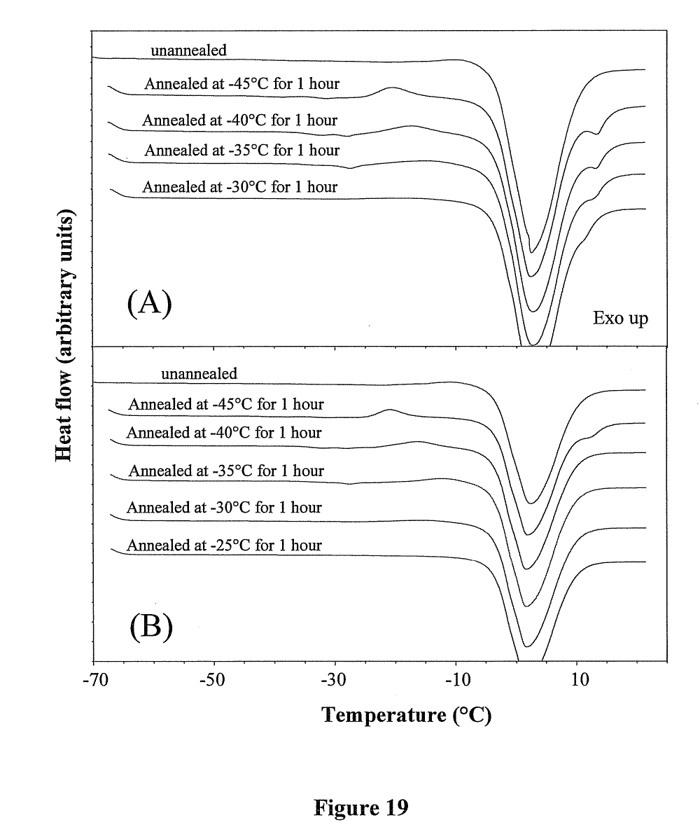

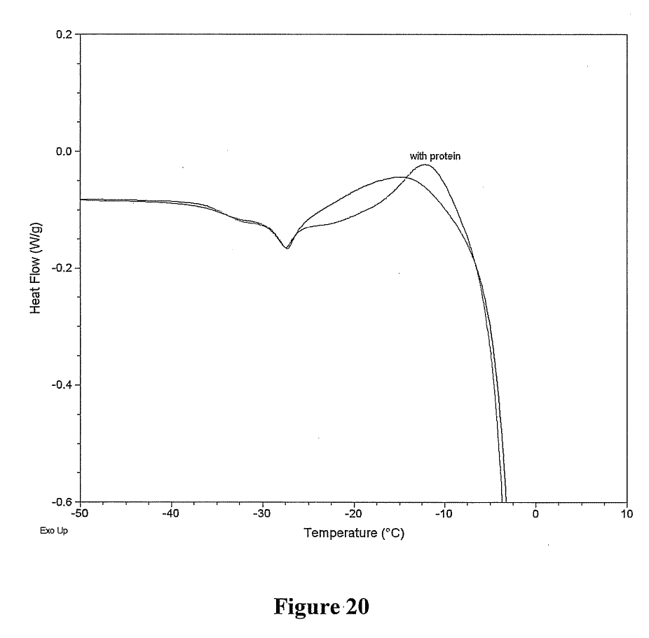

As discussed above, sub-Tg′ annealing facilitated nucleation of mannitol. To determine the effect of the protein when the annealing temperature is higher than the Tg′, the physical stability of the amorphous freeze-concentrate was investigated under more aggressive annealing conditions. The samples were annealed at temperatures ranging from −45, to −25° C. As shown in FIG. 19A, in the absence of the protein, as the annealing temperature increased from −45 to −30° C., the enthalpy of mannitol crystallization, during the second heating decreased. This indicated that during the isothermal annealing, the extent of mannitol crystallization increased as a function of the annealing temperature. Annealing at −30° C. caused complete mannitol crystallization, and as a result, there was no exotherm attributable to mannitol crystallization, during the second heating (FIG. 19A). In the presence of protein, annealing at −30° C. did not cause complete crystallization of mannitol. As a result, crystallization was evident during the second heating (FIG. 19B). However, annealing at a higher temperature of −25° C. caused complete crystallization of mannitol. FIG. 20 also shows the inhibitory effect of the protein on mannitol crystallization. When annealed at −35° C., mannitol crystallization peak was observed immediate after the enthalpy recovery (about −28° C.) in the absence of the protein. On the contrary, in the presence of the protein, mannitol crystallization peak did not emerge until the temperature reached about −22° C. This comparison demonstrated that the protein prevents mannitol crystallization even after annealing at −35° C. (9° C. above the Tg′) for 60 minutes.

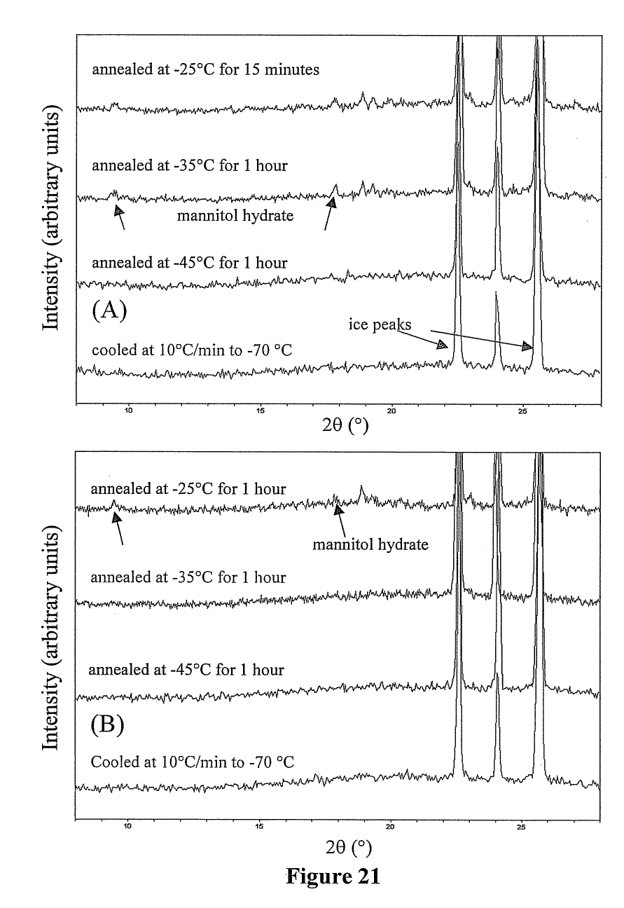

Low-temperature XRD provided direct evidence of the inhibitory effect of the protein on mannitol crystallization. FIG. 21 shows the XRD data of a 5% w/w mannitol solution containing 1.7% w/w sucrose after cooling at 10° C./min to −70° C. and annealing for 60 minutes at different temperatures for both in the absence and presence of the protein. No solute crystallization was detected after cooling to −70° C. In the absence of protein (FIG. 21A), after annealing for 60 minutes at −45° C., mannitol crystallization was not observed. The mannitol hydrate peak (9.4 and 17.9° 2θ) was observed after annealing at −35° C. for an hour. However, the protein was effective in inhibiting mannitol crystallization at this temperature. When the annealing temperature was increased to −35° C., the protein continued to be effective in inhibiting mannitol crystallization. Characteristic peaks (e.g. at 9.1, 18, and 21° 2θ of mannitol hydrate) (see, e.g., Yu et al. 1999. Existence of a Mannitol Hydrate during Freeze-Drying and Practical Implications. Journal of Pharmaceutical Sciences 88: 196-198 emerged only after annealing at −25° C. for 15 minutes).

Significance

The results of this study demonstrate that the active agent, human monoclonal antibody, inhibited mannitol crystallization even under fairly aggressive annealing conditions. The inhibitory effect of human monoclonal antibody was observed at a moderate concentration (20 mg/ml). In addition, this effect was concentration dependent and was more pronounced as the protein concentration was increased (>20 mg/ml).

Conclusions

In summary, the composition of mannitol-sucrose system has an impact on the Tg′. Protein concentration influences the Tg′ and therefore the primary drying temperature. The protein inhibits both the nucleation and crystallization of mannitol. The presence of the protein and the protein concentration also influences the processing conditions (annealing time, annealing temperature and primary drying temperature).

### Example 3

### The Effect of Process Conditions on the Physical State of Mannitol in a Lyophilized Monoclonal Antibody Formulation

The physical state of mannitol was monitored during the various stages of freeze-drying to determine the influence of processing conditions on the physical form of mannitol. The processing conditions investigated were: (1) cooling rate, (2) annealing condition, (3) primary drying, and (4) secondary drying temperatures. A human monoclonal antibody was used as the active agent and mannitol and sucrose were used as the bulking agent and the lyoprotectant, respectively.

Materials

The pre-lyophilized solution used contained 1.7 mg/ml protein, 200 mM mannitol, 60 mM trehalose, 0.01% (w/v) polysorbate 80, and 10 mM phosphate buffer (pH=7.2).

Methods

Differential Scanning Calorimetry (DSC)

A differential scanning calorimeter (MDSC, Model 2920, TA Instruments, New Castle, Del.) with a refrigerated cooling accessory was used. The DSC cell was calibrated using mercury and tin. The samples were prepared in a glove box under nitrogen purge. About 3-6 mg of sample was weighed in an aluminum pan, sealed nonhermetically, cooled from room temperature to −40° C. at 10° C/min and heated to 200° C. under a stream of nitrogen.

About 10-15 mg of the sample solution was weighed in an aluminum pan, sealed hermetically, cooled from room temperature to −70° C. at 20° C./min and maintained at −70° C. for 30 minutes. The frozen solutions were then heated at 5° C./min to room temperature. Only the DSC heating curves were recorded. When there was an annealing step, the frozen solutions were annealed at selected temperature at either −28 or −18° C. for periods of 15 to 60 minutes.

Ambient Temperature X-Ray Powder Diffractometry (XRD)

The powder was filled into an aluminum holder and exposed to CuKα radiation (45 kV×. 40 mA) in a wide-angle X-ray powder diffractometer (Model D5005, Seimens). The instrument was operated in the step scan mode, in increments of 0.04° 2θ. The angle range was 5 to 40° 2θ, and counts were accumulated for 1 second at each step. The data collection program used was JADE 7.0.

Variable Temperature X-Ray Powder Diffractometry (XRD)

An X-ray powder diffractometer (Model XDS 2000, Scintag) with a variable temperature stage (Micristar®, Model 828D, R.G. Hansen & Associates, Santa Barbara, Calif.; working temperature range −190 to 300° C.) was used. An accurately weighed aliquot of sample solution (˜100 mg) was filled into a copper holder and cooled from room temperature to −45° at 1° C./min. The frozen solutions were then held for 15 minutes, heated at 1° C./min to the desired annealing temperature, and then held for 3 hours (XRD patterns were obtained at regular intervals). XRD patterns were obtained by exposing the sample to CuKα radiation (45 kV×. 40 mA), wherein the step size was 0.04° 2θ. The angular range was 5 to 30° 2θ and counts were accumulated for 1 second at each step. The samples were maintained under isothermal conditions at the selected temperatures. After annealing, the frozen solutions were then subjected to primary drying in situ in the sample chamber of the XRD at a pressure of ˜100 mTorr. Primary drying was carried out until all the crystalline ice was sublimated. The sample was then heated to the selected secondary drying temperatures where the drying was continued for the desired time period.

Results and Discussion

Solid State Characterization of Final Lyophiles

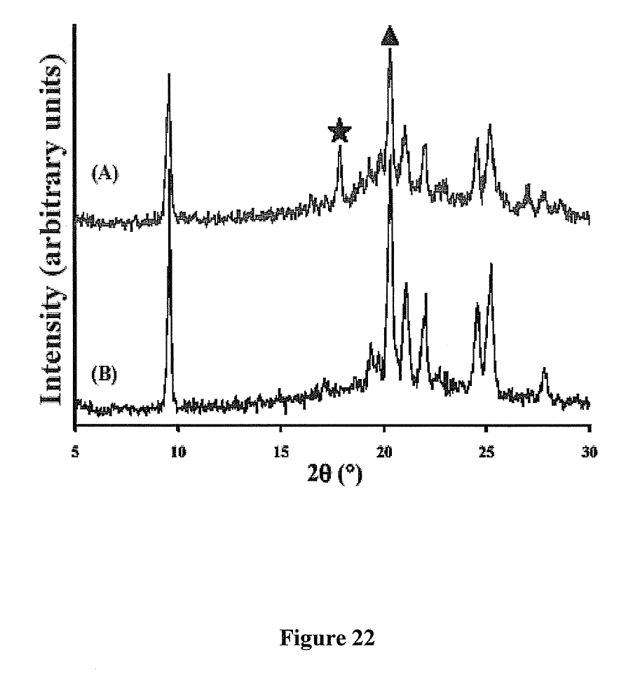

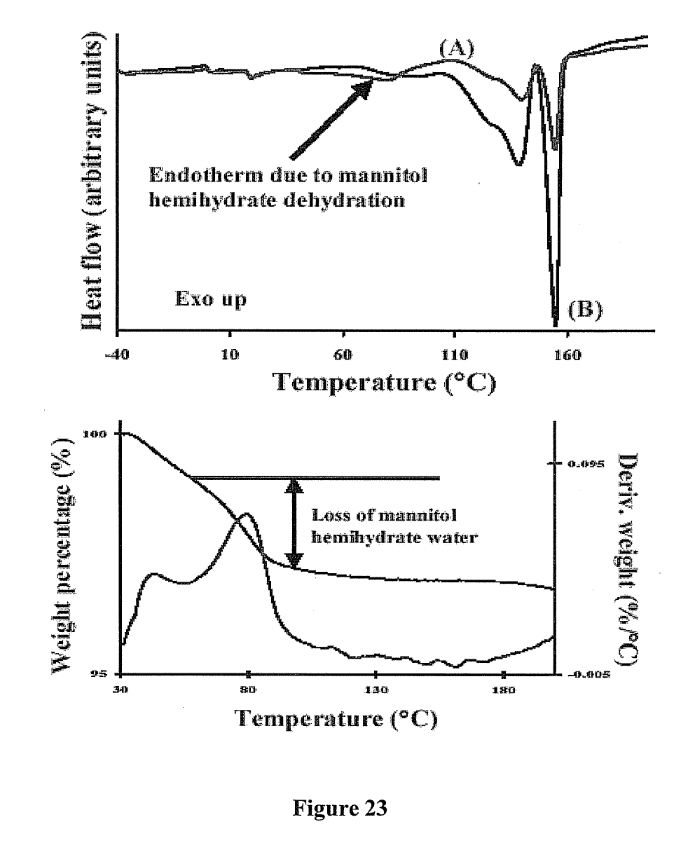

The lyophilization conditions influenced the physical form of mannitol in the final lyophiles. Prelyo solution was freeze-dried under two conditions, and the lyophiles were characterized by several techniques. While the presence of δ-mannitol (characteristic peak at 9.6° 2θ) was evident in both the lyophiles, the mannitol hemihydrate content in sample A (characteristic peak at 17.9° 2θ) was higher than in sample B (FIG. 22). This was also evident from DSC, wherein a pronounced endotherm at ˜80° C., attributable to dehydration of the hemihydrate, was observed only in sample A (FIG. 23). The amorphous nature of the dehydrated phase was evident from the crystallization exotherm, which followed the dehydration endotherm. The weight loss in the TGA occurred in two stages, with the first step attributable to the loss of sorbed water and the second (over the temperature range of ˜70 to 85° C.) to dehydration (FIG. 23). The above observation indicated that processing conditions affect the physical state of mannitol in the final lyophiles. In order to identify the processing parameter(s) that influence the physical state of mannitol, each stage of the freeze-drying cycle was monitored starting with the frozen system.

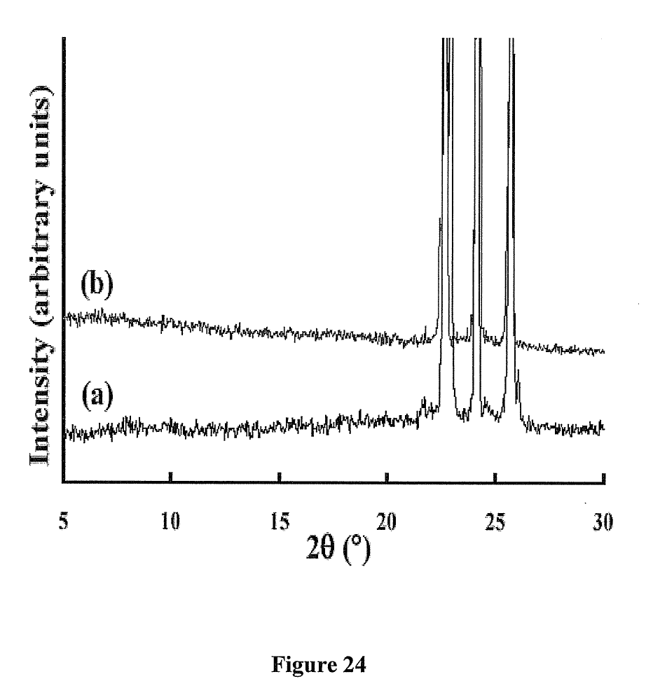

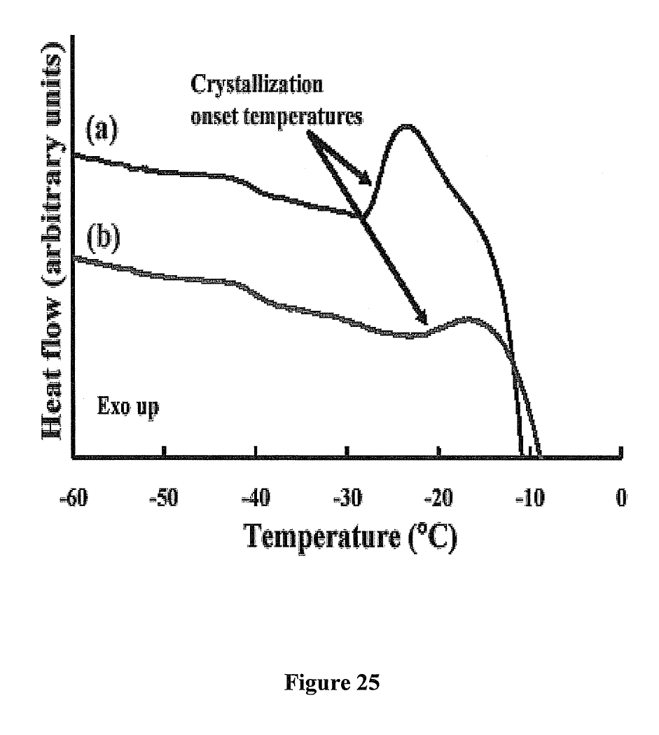

The prelyo solution was either quench-cooled with liquid nitrogen or cooled to −70° C., at controlled rates ranging from 1 to 10° C./min (FIG. 24). Crystalline mannitol was not detected in the frozen solution. This conclusion was based on the XRD patterns of the frozen solution (two representative patterns are shown in FIG. 24). Irrespective of the cooling rate, when the frozen solution was heated at 5° C./min to 25° C., the following thermal events were observed: (i) glass transitions with onset at ˜−41° C. and ˜−32° C., (ii) an exotherm with onset at ˜−26° C., ascribed to mannitol crystallization, and (iii) overlapping eutectic and ice melting endotherms (FIG. 25). The observed Tg′ values were in reasonable agreement with the reported values of −41° C. and −33° C. in systems with similar but not identical compositions. See, Pyne et al., Pharm Res. 19:901-908 (2002). A placebo solution, subjected to the same treatment, exhibited similar Tg′ values (FIG. 25). Thus, the Tg′ values seemed to be unaffected by the presence of the protein which may be due to the fact that the protein concentration in the prelyo solution was low (1.7 mg/ml). However, the crystallization onset temperature was shifted to a lower temperature (−22.6 to −25.7° C.) in the presence of protein suggesting that the protein facilitated mannitol crystallization.

Effect of annealing

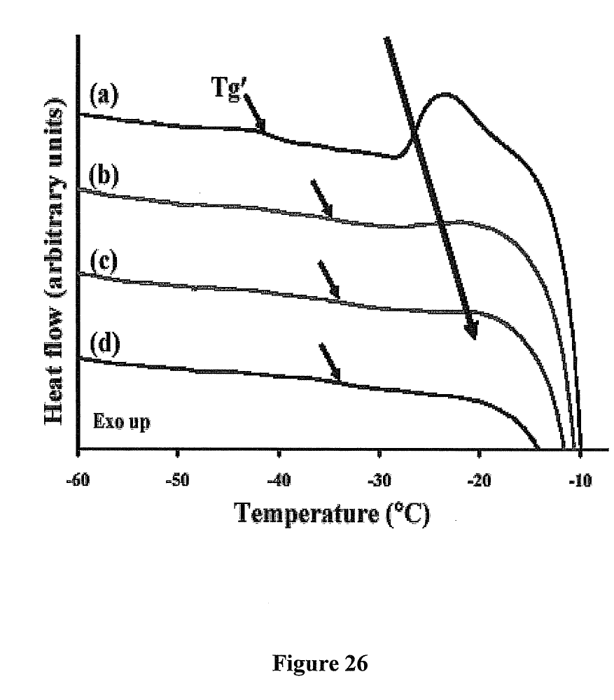

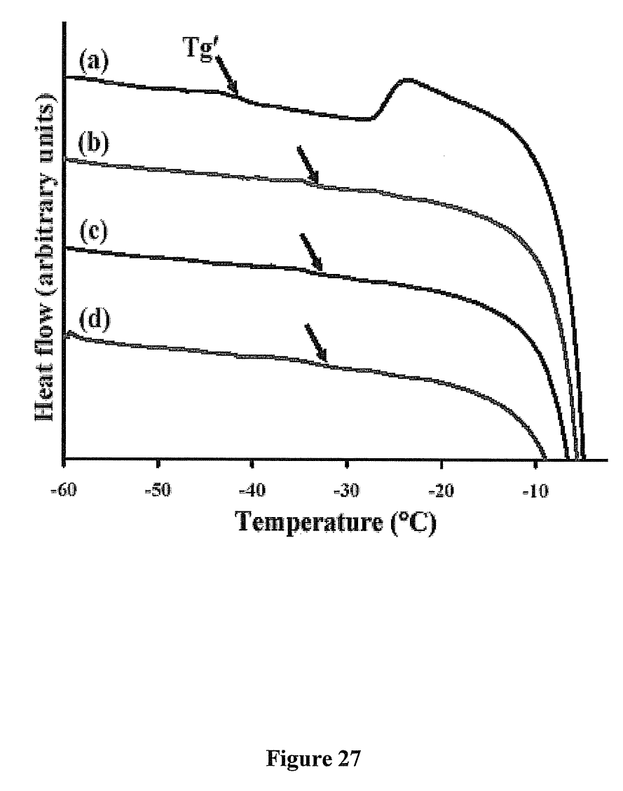

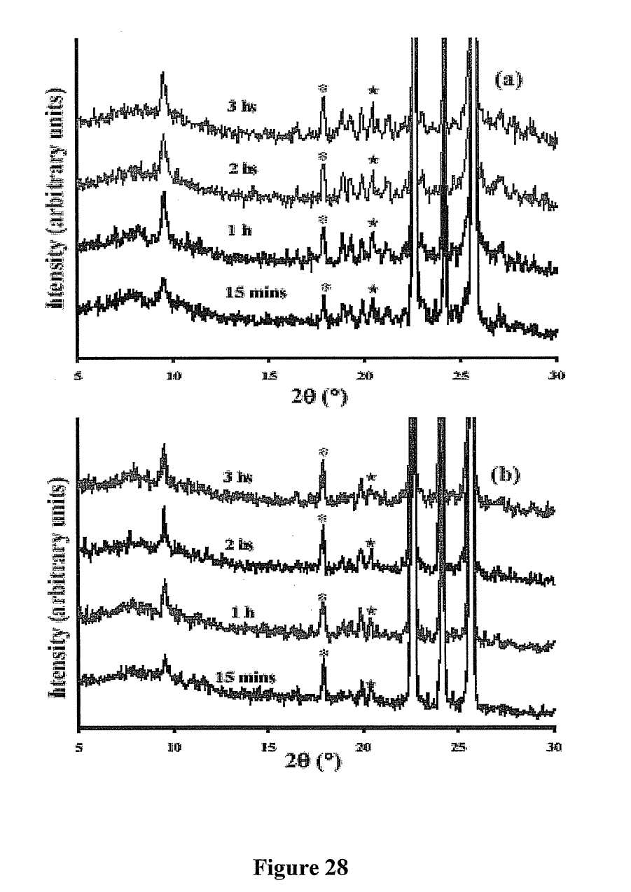

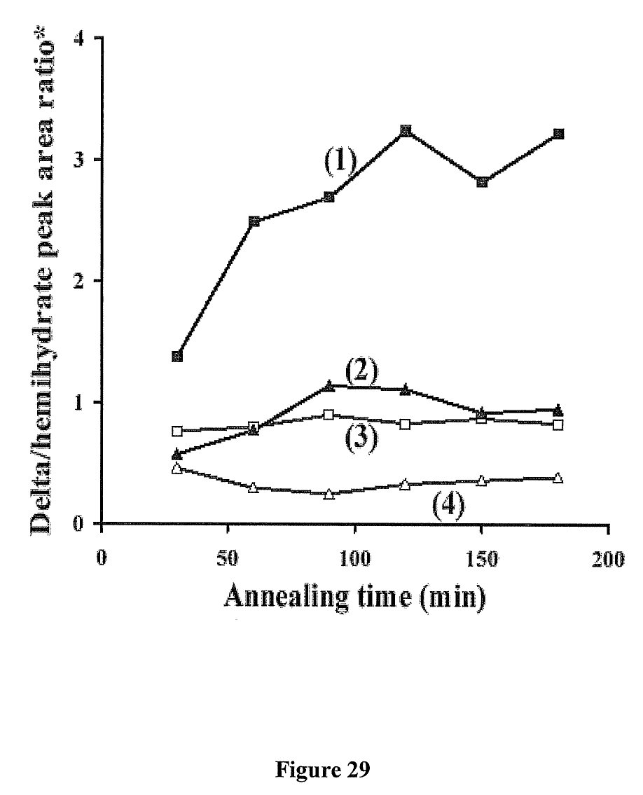

Annealing, an isothermal step to promote solute crystallization, is commonly conducted at temperatures above Tg′. Two temperatures, −28° C. and −18° C., were chosen for annealing. When annealed at −28° C., crystallization enthalpy gradually decreased as the annealing time increased from 15 minutes to 45 minutes (FIG. 26). The crystallization peak was not observed when the annealing time reached 60 minutes. On the other hand, crystallization was rapid when annealing was conducted at −18° C., and was complete in 15 minutes (FIG. 27). Irrespective of the annealing temperature, there was a shift in Tg′ to a higher temperature of −32° C. XRD was then used to identify the phases crystallizing from solution. The solutions were cooled to −45° C. (at 1° C./min), held for 15 minutes, and heated at 1° C./min to the desired annealing temperature, and held for 3 hours, and XRD patterns were obtained at regular intervals. There was no detectable solute crystallization in the unannealed frozen solutions. After annealing for 15 minutes at −18° C., solute crystallization, both as mannitol hemihydrate and as δ-mannitol was observed (FIG. 28(a) and 28(b)). As annealing time increased, the intensity of the characteristic peaks of both mannitol hemihydrate and δ-mannitol increased. In order to obtain a semi-quantitative trend of the mannitol phases during annealing, the integrated intensity ratio (intensity of 20.4° 2θ peak of δ-mannitol/intensity of 17.9° 2θ peak of mannitol hemihydrate) was plotted as a function of annealing time (FIG. 29). The presence of protein seemed to selectively inhibit the formation of mannitol hemihydrate. In order to determine if this inhibitory effect was temperature dependent, annealing was also conducted at a higher temperature of −8° C.

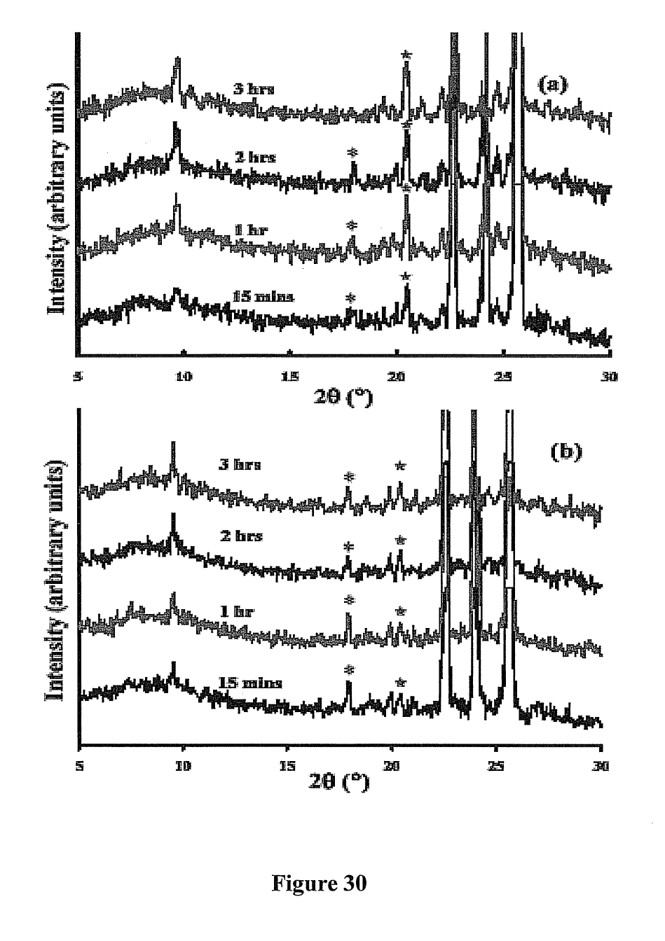

As before, after annealing for 15 minutes at −8° C. mannitol hemihydrate and δ-mannitol crystallized, both in the presence and absence of protein (FIGS. 30(a) and 30(b)). The inhibitory effect of protein on mannitol hemihydrate was evident at longer annealing times. There was an increase in the δ-mannitol peak intensity (20.4° 2θ) at the expense of mannitol hemihydrate (FIG. 29, curve (1); FIG. 30(a)). After 3 hours of annealing, the characteristic peak of mannitol hemihydrate had almost disappeared (FIG. 30(a)). In the placebo, both mannitol phases were still present after 3 hours of annealing (FIG. 30(b)). The DSC experiments showed that the crystallization onset temperature was lowered in the presence of protein, suggesting that the protein facilitated mannitol crystallization (FIG. 25). This was also confirmed by XRD, wherein the intensity of mannitol peaks (both δ- and hemihydrate) was observed to be higher in the presence of protein (FIGS. 28(a) and 28(b)). XRD revealed the additional information that the protein facilitated formation of δ-mannitol possibly at the expense of mannitol hemihydrate.

Effect of Primary Drying

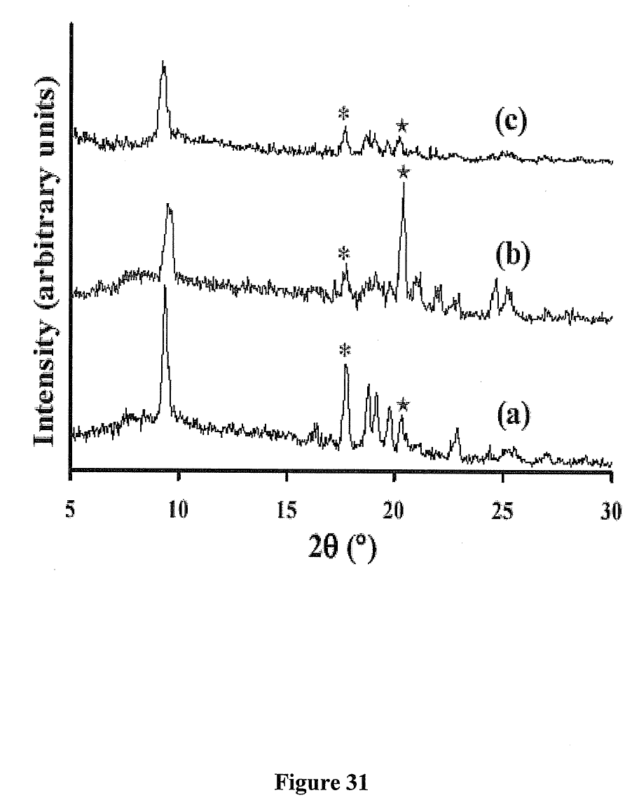

By attaching a vacuum pump to the low-temperature stage, the freeze-drying cycle was simulated in the sample chamber of the X-ray diffractometer. When the solution annealed at −8° C. was primary dried at −5° C., which is slightly below the eutectic temperature, there was an increase in the intensity of δ-mannitol peaks (FIG. 31(b)). The solution annealed at −8° C. contained low amounts of mannitol hemihydrate (FIG. 30a), and the primary drying also seemed to favor the formation of δ-mannitol. Thus, even if mannitol hemihydrate was formed during primary drying, it may be unstable at the high primary drying temperature, and may be transformed to δ-mannitol. In order to test this possibility, primary drying was conducted at a lower temperature of −20° C. The presence of the mannitol hemihydrate peak suggests that the hemihydrate phase can be retained at low primary drying temperatures (FIG. 31(c)). Annealing at −18° C. had resulted in a mixture of the anhydrous and hemihydrate phases (FIG. 28(a)). While both the phases were retained on primary drying at −5° C., the hemihydrate content was higher in the final lyophile (FIG. 31(a)). Thus, the concentration of the hemihydrate in the frozen solution increased the retention of the hemihydrate in the final lyophile, even when the primary drying temperature was high. These results suggest that if the hemihydrate is not desired in the final lyophile, it should be removed before initiating the primary drying.

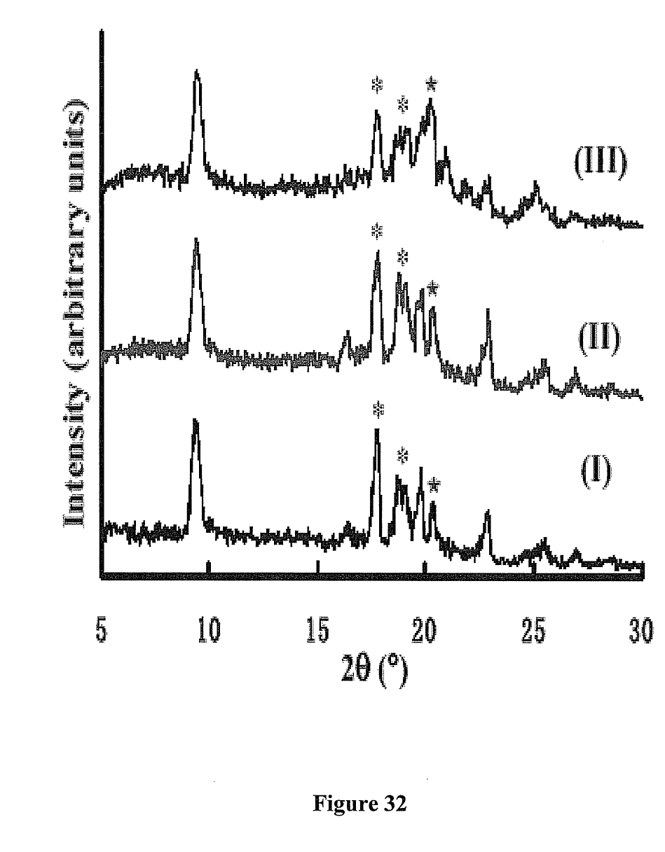

Once mannitol hemihydrate was formed, it was only reduced from the final lyophile when secondary drying was conducted at a high temperature. FIG. 32 contains the XRD patterns of lyophile secondary dried at progressively higher temperatures up to 65° C. While secondary drying at 25 and 45° C. did not cause dehydration, drying at 65° C. for 30 minutes caused partial dehydration and possibly conversion to δ-mannitol. The resistance to dehydration in the presence of protein was earlier reported by Johnson et al., J. Pharm. Sci. 91:914-922 (2002), while preparing a freeze-dried formulation of daniplestim (a recombinant protein) containing sucrose and mannitol. A shelf temperature of 40° C. and a drying time of 13 hours were the mildest secondary drying conditions for elimination of mannitol hemihydrate.

Significance

The above observation has several implications. Traditionally, solid-state and physical characterization studies have been restricted to the final freeze-dried product. This does not provide information about physical changes (glass transition, crystallization, and solid-state transition) during the various stages of the freeze-drying process. The experiment investigated the crystallization behavior of mannitol in the presence and absence of the protein during the various stages of the freeze-drying process. The results indicate that the physical state of mannitol was influenced by the processing conditions as well as the formulation components including the API.

Conclusions

The results show the effect of processing conditions on the phase behavior of mannitol in a protein formulation during freeze-drying. Mannitol did not crystallize during cooling even at a slow cooling rate of 1° C./min. Annealing facilitated mannitol crystallization. In the absence of the protein, elevating annealing temperature did not change the outcome of physical form of mannitol. In the presence of protein, high annealing temperature promoted delta mannitol crystallization and inhibited formation of mannitol hemihydrate, while low annealing temperatures facilitated the formation of mannitol hemihydrate. Mannitol hemihydrate can survive even at a relatively high secondary drying temperature (45° C.), but there was evidence of dehydration at 65° C. Therefore, the solid state of mannitol in the final lyophile appeared to be a complex function of annealing conditions, primary drying and secondary drying temperatures.

TABLE 2
Compositions of mannitol-sucrose mixture solutions

| Mannitol to | Mannitol | Sucrose | Citrate buffer |
|---|---|---|---|
| sucrose ratio (R) | (% w/w) | (% w/w) | (% w/w) |
| 0.45 | 2.41 | 5.00 | 92.60 |
| 1.10 | 3.54 | 3.21 | 93.30 |
| 1.50 | 4.03 | 2.70 | 93.30 |
| 1.95 | 4.16 | 2.12 | 93.70 |
| 2.50 | 5.00 | 2.50 | 92.50 |
| 3.00 | 5.00 | 1.67 | 93.20 |

What is claimed is:

1. A liquid formulation suitable for freeze-drying to form a freeze-dried formulation, the liquid formulation comprising:
a protein active agent;
a saccharide stabilizing agent; and
a bulking agent,
wherein the weight ratio of bulking agent to saccharide stabilizing agent and protein active agent in the liquid formulation is sufficient to maintain the bulking agent in a substantially amorphous state wherein less than about 25 wt. % of the bulling agent present in the formulation is in a crystalline state.

2. The liquid formulation of claim 1, wherein the protein active agent is included in the liquid formulation at a concentration between about 0.1 mg/ml and about 100 mg/ml.

3. The liquid formulation of claim 1, wherein the ratio of bulking agent to saccharide stabilizing agent is between about 5:1 and about 0.2:1.

4. The liquid formulation of claim 1, further comprising a surfactant.

5. The liquid formulation of claim 1, wherein the bulking agent comprises mannitol.

6. The liquid formulation of claim 1, wherein the saccharide is a disaccharide.

7. The liquid formulation of claim 1, wherein the protein active agent is an antibody.

8. The liquid formulation of claim 1, wherein the protein active agent is a non-antibody protein.

9. The liquid formulation of claim 8, wherein the protein is a fusion protein.

10. The liquid formulation of claim 1, wherein the freeze-dried formulation is for subcutaneous administration

11. The liquid formulation of claim 1, wherein the freeze-dried formulation is for intravenous administration.

12. A process for preparing a freeze-dried formulation, comprising the steps of:
(a) preparing a liquid formulation comprising:
(i) a protein active agent;
(ii) a saccharide stabilizing agent; and
(iii) a bulking agent,
(b) freezing the liquid formulation to form a frozen formulation under conditions sufficient to maintain the bulking agent in a substantially amorphous state wherein less than about 25 wt % of the bulking agent present in the formulation is in a crystalline state;
(c) drying the liquid formulation to form a freeze-dried formulation, wherein the drying step is performed under conditions sufficient to maintain the bullring agent in a substantially amorphous state wherein less than about 25 wt % of the bulking agent present in the formulation is in a crystalline state.

13. The process of claim 12, wherein the protein active agent is included in the liquid formulation at a concentration between about 0.1 mg/ml and about 100 mg/ml.

14. The process of claim 12, wherein the ratio of bulking agent to saccharide stabilizing agent is between about 5:1 and about 0.2:1.

15. The process of claim 12, wherein the liquid formulation further comprises a surfactant.

16. The process of claim 12, wherein the bullring agent comprises mannitol.

17. The process of claim 12, wherein the saccharide stabilizing agent comprises a disaccharide.

18. The process of claim 12, wherein the protein active agent is an antibody.

19. The process of claim 12, wherein the protein active agent is a non-antibody protein.

20. The process of claim 19, wherein the protein is a fusion protein.
